(19) United States
(12) Patent Application Publication
(10) Pub. No.: US 2024/0067717 A1
(43) Pub. Date: Feb. 29, 2024

(54) DIAGNOSIS AND TREATMENT OF PRIMARY GRAFT DYSFUNCTION FOLLOWING LUNG TRANSPLANT

(71) Applicant: Northwestern University, Evanston, IL (US)

(72) Inventors: Ankit Bharat, Evanston, IL (US); Emilia Lecuona, Evanston, IL (US); Wenbin Yang, Evanston, IL (US)

(73) Assignee: Northwestern University, Evanston, IL (US)

(21) Appl. No.: 18/240,201

(22) Filed: Aug. 30, 2023

Related U.S. Application Data
(60) Provisional application No. 63/402,872, filed on Aug. 31, 2022.
(60) Provisional application No. 63/414,345, filed on Oct. 7, 2022.

(51) Int. Cl. C07K 16/24; A61K 38/20; A61P 37/06; G01N 33/68

(52) U.S. Cl. CPC C07K 16/245; A61K 38/2006; A61P 37/06; G01N 33/689; C07K 2317/21; G01N 2333/545; G01N 2800/245; G01N 2800/50

(57) ABSTRACT

Provided herein are methods of preventing or reducing the incidence of primary graft dysfunction in a subject.

### CROSS-REFERENCE TO RELATED APPLICATIONS

This application claims the benefit of priority to U.S. Provisional Patent Application No. 63/402872, filed on Aug. 31, 2022, and 63/414,345, filed on Oct. 7, 2022, the contents of which are incorporated herein by reference in their entirety.

### STATEMENT OF GOVERNMENT SUPPORT

This invention was made with government support under grant 5R01HL147290-04 awarded by the National Institutes of Health (NIH). The government has certain rights in the invention.

### TECHNICAL FIELD

The present application relates to treatment of primary graft dysfunction following lung transplant.

### BACKGROUND

Primary graft dysfunction (PGD) is a lethal complication after lung transplantation with unclear etiology. It has been reported that over 30-50% of patients undergoing lung transplantation have pre-existing lung autoantibodies that are associated with increased risk of PGD. However, it was unknown whether the lung autoantibodies are causally linked to PGD. Additionally, it was unclear what through what mechanisms the autoantibodies cause PGD.

### SUMMARY

The Applicants have discovered that a chemokine IL1β that is released by circulating monocytes and potentially other cells may allow lung autoantibodies to leak from the blood vessels into the interstitial space of the lung where they bind to the self-proteins. This binding of the autoantibodies to the proteins may activate the classical and alternate complement pathways and result in the destruction of the newly transplanted lung. Also, there are potential tests that can be used to monitor for these autoantibodies and make the clinical decision of when to treat.

The present disclosure indicates that existing commercially available tests have a new indication to screen all patients undergoing lung transplantation to detect autoantibodies in order to prevent PGD. Therefore, there are several indications that can prevent or treat the damaging effects of these autoantibodies on the new lungs. The following disclosure validates these findings in a pilot clinical study and in the form of case studies.

An embodiment is a method of preventing or reducing the incidence of primary graft dysfunction in a subject, comprising measuring a level of lung restricted autoantibodies in the subject and administering an anti-IL1β antibody and/or complement inhibitor to the subject if lung restricted autoantibodies are determined to be present above a threshold level. Preferably, the subject is a human being.

Another embodiment is a method of determining the likelihood of primary graft dysfunction in a subject, comprising detecting the presence of chemokine IL1β in a sample from the subject following lung transplantation.

One aspect of the present disclosure includes the use of the existing Luminex test (autoantibody multiplex assay) to screen all patients who need lung transplantation. An example of an existing Luminex test includes LABScreen autoantibody kits (Cat #LSAUT1-3, One Lambda).

A further aspect of the present disclosure includes the use of anti-IL1β antibodies or anti-IL1 receptor antibodies to treat patients with these autoantibodies to prevent or treat PGD. In some aspects, the anti-IL1β antibodies include Canakinumab, also known by the trade name Ilaris. In some aspects, the anti-IL1 receptor antibodies include Anakinra, also known by the trade name Kineret. In some aspects, the subject is screened for the presence of lung autoantibodies prior to administration of the anti-ILβ antibodies or the anti-IL1 receptor antibodies.

A further aspect of the present disclosure includes the use of complement inhibitors to treat PGD in the presence of autoantibodies. In some aspects, the complement inhibitors include Eculizumab, Cinryze, or Rocunest among others.

In yet another aspect of the present disclosure, a method for treating PGD is provided. The method includes screening the subject for the presence of lung restricted antibodies and administering an anti-IL1β antibody and/or complement inhibitor. In one aspect, the screening step comprises a Luminex test to screen the subject for the lung restricted antibodies.

In yet another aspect of the present disclosure, a method of treating or preventing primary graft dysfunction in a subject is provided. In one aspect, the method includes administering an anti-lung restricted antibody. In one aspect, the anti-lung restricted antibody includes an anti-IL1β antibody. In yet another aspect, wherein the anti-IL1β antibody comprises Canakinumab or Anakinra or derivatives thereof.

In yet another aspect of the present disclosure, a method of treating or preventing primary graft dysfunction in a subject is provided. In one aspect, the method includes administering a complement inhibitor. In one aspect, the complement inhibitor includes Eculizumab, Cinryze, or Rocunest, or derivatives thereof.

In yet another aspect of the present disclosure, a method of determining the likelihood of primary graft dysfunction in a subject is provided. In one aspect, the method includes detecting the presence of chemokine IL1β in a sample from the subject following lung transplantation.

In yet another aspect of the present disclosure, a method of preventing or reducing the incidence of primary graft dysfunction in a subject is provided. In one aspect, the method includes screening the subject for the presence of lung restricted antibodies and administering an anti-IL1β antibody and/or complement inhibitor to the subject. In one aspect, the subject is a human being.

In yet another aspect of the present disclosure, a method for identifying transplantation patients who are likely to benefit from IL1β therapy through autoantibody testing is provided. In one aspect, a method for selecting patients as candidates for IL1β therapy includes screening a patient to determine whether the patient exhibits autoantibodies against the lungs prior to transplantation. In one aspect, the screening is performed using any autoantibody testing technique, including, but not limited to, Enzyme-Linked Immunosorbent Assay (ELISA), LUMINEX, multiplex immunoassay, or a commercially available test, such as those provided by One Lambda.

In yet another aspect of the present disclosure, a method for targeting IL1β or its receptor IL1R to mitigate the harmful effects of autoantibodies in a patient is provided. In one aspect, the method includes administering to a patient an antibody against IL1β or IL1R, thereby neutralizing the impacts of IL1β.

In yet another aspect of the present disclosure, a method for administering an IL1β-targeting antibody to a patient is provided. In one aspect, the IL1β-targeting antibody is administered at least 30 minutes prior to the reperfusion of the transplanted lungs. In one aspect, administration occurs post skin incision on the recipient at the commencement of the operation. In one aspect, in the case of substantial blood loss (e.g., exceeding 500 cc) during the old lung dissection and before the new lung implantation, at least one additional dose of the IL1β-targeting antibody is administered to the patient.

In one aspect, the IL1β-targeting antibody is a monoclonal or a polyclonal antibody against IL1β. In one aspect, the at least one additional dose is a single dose. In one aspect, more than one dose may be administered. As a non-limiting example, a single dose at 24 hours post-transplantation may be administered to the patient, and again at 48 hours if there is ongoing blood loss following the procedure or if primary graft dysfunction is observed.

In one aspect, IL1R may be employed to counteract IL1 effects. In one aspect, an antibody against IL1R is administered to the donor during organ preservation, prior to donor heart cross-clamping. In one aspect, the antibody against IL1R is administered through intravenous injection post skin incision in the donor. In one aspect, the antibody against IL1R is administered as part of the preservative solution during lung perfusion post cross-clamping, or during ex vivo lung perfusion. In one aspect, a second dose of the antibody against IL1R, either intravenously or subcutaneously, may be administered to the recipient within 24 hours for enhanced effectiveness.

### BRIEF DESCRIPTION OF THE DRAWINGS

FIGS. 1A-1F. Preexisting whole lung-restricted antibodies (LRA) against self-antigens induces primary graft dysfunction after syngeneic murine lung transplantation. Recipient mice received 150 μg each (i.v.) of isotype control, LRA (collagen type V plus K-a.1 tubulin), or LRA F(ab′)₂ portion, 24 hours before and 1 hour after lung transplantation. FIG. 1A Shows arterial blood oxygenation analysis after clamping the right hilum at 24 hours post-transplant (n=3-4). FIG. 1B shows assessment of pulmonary edema in harvested lungs (n=3-5). FIG. 1C shows neutrophil counts (live CD45+ SiglecF-CD11b+Ly6G+) (n=3). FIG. 1D shows histology of capillaritis and alveolar edema in mice treated with LRA but not isotype control antibodies. FIG. 1E shows inflammatory cells (both polymorpho- and mononuclear) counted in 10 high-power fields (40×) and averaged for each group. Bar: 20 μm. FIG. 1F shows pictures from two photon microscopy showing LRA (magenta) deposition. Bar: 50 μm. Data are presented as mean±SD. PaO₂/FiO₂, arterial oxygen pressure. FIGS. 1A-C were analyzed by one-way Anova followed by Tukey's post-hoc test. FIG. 1E was analyzed by Unpaired Student's t-test. *p<0.05; **p<0.01; ***p<0.001; ****p<0.0001.

FIGS. 2A-2C. Differential expression analysis of single-cell RNA-Seq data from isotype or LRA-treated murine allograft after lung transplantation within epithelial cells. FIG. 2A shows functional enrichment analysis with GO Biological Processes performed with the significantly upregulated genes in epithelial cells from LRA-treated compared with isotype-treated murine lung allograft. FIGS. 2B and 2C show violin plots of expression for select genes significantly upregulated in epithelial cells from LRA-treated compared with isotype-treated murine lung allograft.

FIGS. 3A-3E. LRA activate complement pathway to mediate lung graft injury. FIG. 3A shows WT or Fcry−/−recipients received 150 μg each (i.v.) of isotype control or LRA (collagen type V plus K-α1 tubulin) 24 hours before and 1 hour after lung transplantation. Arterial blood oxygenation was analyzed after clamping the right hilum (n=4). FIG. 3B shows lungs treated as in FIG. 3A were also harvested for neutrophil quantification (live CD45+ SiglecF-CD11b+Ly6G+) (n=3). FIG. 3C shows WT or Fcry^{-/-} recipient mice treated as in FIG. 3A, 24 hours post-transplant, bronchoalveolar lavage fluid (BALF) was obtained and C3 concentration measured by ELISA (n=3-6). FIG. 3D shows WT or C3^{-/-} recipient were treated as in FIG. 3A, arterial blood oxygenation was analyzed after clamping the right hilum (n=3). FIG. 3E shows quantification of neutrophil recruitment into lungs in mice treated as in FIG. 3A (neutrophils gating: live CD45+ SiglecF-CD11b+Ly6G+) (n=3-5). Data are presented as mean±SD. PaO₂/FiO₂, arterial oxygen pressure. Graphs were analyzed by one-way Anova followed by Tukey's post-hoc test. *p<0.05; **n<0.01; ***n<0.001; ****n<0.0001; n.s. not significant.

FIGS. 4A-4E. LRA activate classical and alternative complement pathway to mediate lung graft injury. FIG. 4A shows a diagram depicting experiments shown in FIGS. 4B and C. WT, C1q−/− or Mb1^{-/-} recipient mice received 150 μg each (i.v.) of isotype control or LRA (collagen type V plus K-α1 tubulin) 24 hours before and 1 hour after lung transplantation. For some experiments WT recipient mice treated with antibodies as described received C1INH, LNPO23 (Factor B inhibitor, FBI) or anti-C5 neutralizing antibody. FIG. 4B shows arterial blood oxygenation from mice described in FIG. 4A was analyzed after clamping the right hilum (n=3). FIG. 4C shows quantification of neutrophil recruitment into lungs in mice treated as described in FIG. 4A (neutrophils gating: live CD45+ SiglecF-CD11b+Ly6G+) (n=3-5). FIGS. 4D and 4E show Immunocytochemistry for C4d (FIG. 4D) and C5b-9 (FIG. 4E) in allografts of LRA-treated mice (right) compared with Isotype-treated mice (left). Data are presented as mean±SD. PaO₂/FiO₂ arterial oxygen pressure. Graphs were analyzed by one-way Anova followed by Tukey's post-hoc test. *p<0.05; ***p<0.001; ****p<0.0001; n.s. not significant. Bar: 20 μm. Inset bar: 10 μm.

FIGS. 5A-5E. LRA-induced lung dysfunction is complementary to ischemia-reperfusion injury. FIG. 5A shows a diagram depicting experiment shown in FIGS. 5B and C. WT recipient mice received 150 μg each (i.v.) of isotype control or LRA (collagen type V plus K-a.1 tubulin) 24 hours before and 1 hour after lung transplantation using donor Nr4al−/− mice. FIG. 5B shows arterial blood oxygenation from mice described in (A) was analyzed after clamping the right hilum (n=3). FIG. 5C shows quantification of neutrophil recruitment into lungs in mice treated as described in FIG. 5A (neutrophils gating: live CD45+ SiglecF-CD11b+Ly6G+) (n=3-4). FIG. 5D shows WT recipient mice treated as in FIG. 5A were injected (i.p.) with isotype or anti-Ly6G antibody 24 hours before lung transplant and harvested for determination of pulmonary edema at 24 hours after transplant (n=3). FIG. 5E shows WT recipient mice treated as described in FIG. 5D. Arterial blood oxygenation was analyzed after clamping the right hilum (n=3-4).Data are presented as mean±SD. PaO₂/FiO₂ arterial oxygen pressure. Graphs were analyzed by one-way Anova followed by Tukey's post-hoc test.*p<0.05; **p<0.01; ***p<0.001; ****p<0.0001; n.s. not significant.

FIGS. 6A-6G. IL1β increases vascular endothelial permeability and extravasation of LRA. FIG. 6A shows mouse primary lung microvascular endothelial cells seeded at 200,000 cells per insert and cultured until confluence. Monolayers were incubated for 24 hours in the absence or presence of 50 ng/mL IL1β in growth medium. FITC-antibody permeability testing was performed as described in Methods. FIG. 6B shows a diagram depicting experiment shown in FIGS. 6C and D. WT recipient mice received 150 μg each (i.v.) of isotype control or LRA (collagen type V plus K-a.1 tubulin) 24 hours before and 1 hour after lung transplantation. Donor lungs were from Il1r−/− mice or WT mice treated with IL1RA 24 hours before and 1 hour after lung transplant. FIG. 6C shows arterial blood oxygenation from mice described in FIG. 6B was analyzed after clamping the right hilum (n=3). FIG. 6D Quantification of neutrophil recruitment into lungs in mice treated as described in FIG. 6B (neutrophils gating: live CD45+ SiglecF-CD11b+Ly6G+) (n=3-5). FIGS. 6E-G shows immunohistochemistry for C4d FIGS. 6E and G and LRA FIG. 6F in allografts of LRA-treated wild-type mice (upper) compared with LRA-treated I11r−/− mice (lower) (FIG. 6E) or in lungs from PBS- or IL1B-treated mice (FIGS. 6F and G). Data are presented as mean±SD. PaO₂/FiO₂, arterial oxygen pressure. Graphs were analyzed by one-way Anova followed by Tukey's post-hoc test. *p<0.05; **p<0.01; ***n<0.001. Bar: 20 μm.

FIG. 7 shows deposition of LRA in native lungs and lung allografts. PE-labelled Col-V antibodies were injected in control wild-type mice or murine recipients 24 hours after lung transplant. Two photon imaging was performed during the described time-course. Magenta: LRA. Green: Blood vessels. Bar: 50 μm.

FIG. 8 shows single cell RNA sequencing from LRA and isotype control treated murine recipients. Mice in both groups were treated as described in the methods and then underwent syngeneic transplants. At 24 hours following transplantation, grafts were harvested, and single cell RNA sequencing performed. The figure demonstrates the cell map.

FIGS. 9A-9B show anti-Ly6G antibody efficiently depleted neutrophils. WT recipient mice received 150 μg each (i.v.) of isotype control or LRA (collagen type V plus K-α1 tubulin) 24 hours before and 1 hour after lung transplantation as well as isotype or anti-Ly6G antibody 24 hours before lung transplant. FIG. 9A shows histology showing neutrophil and mononuclear infiltration (arrowhead) in mice treated with isotype but not in anti-Ly6G antibody treated mice. FIG. 9B shows quantification of neutrophil recruitment into lungs (neutrophils gating: live CD45+ SiglecF-CD11b+Ly6G+) (n=3). Data are presented as mean±SD. Graph was analyzed by Unpaired Student's t-test. **p<0.01. Bar: 20 μm. chest radiograph and histology in a patient with pre-existing LRA who developed PGD.

FIGS. 10A-10B show patients with pre-existing LRA who developed PGD showed signs of antibody mediated rejection. A representative patient is illustrated. FIG. 10A shows chest radiographs showing progressive PGD on post-operative days 1 (i) and 3 (ii) as well as improvement after 2 (iii) and 7 (iv) days of initiation of plasmapheresis and complement inhibition. FIG. 10B shows histological analysis of intraoperative post-reperfusion biopsy showing: (i) Lung parenchyma with fibrin, red blood cells and scattered neutrophils (20×), (ii) reactive alveolar pneumocytes with acute and chronic inflammatory cells (20×), (iii) capillary congestion (20×), and (iv) complement C4d staining. The donor lung prior to reperfusion appeared normal (v).

FIGS. 11A-11B show that the release of IL1β increases the extravasation of LRA, promoting its binding to cognate antigens outside the blood vessels. It was previously demonstrated that IL1β facilitates the opening of endothelial gap junctions. This, in turn, enables the extravasation of LRA circulating in the blood. In FIGS. 11A-11B, LRA was administered to mice, which then underwent lung transplantation. At the various time points listed above, immunohistochemistry and immunofluorescence were conducted to measure the levels of LRA deposited in the lung tissue. In FIG. 11A, LRA (colored pink) was observed to be deposited outside the blood vessels, consistent with prior findings that lung transplantation is associated with the release of ILβ. To further validate the role of IL1β, LRA was administered to normal mice. These mice, now containing LRA, were then given either IL1β or PBS (as a control). Injection with IL1β led to the extravasation of LRA, while PBS showed no effect. FIG. 11B presents the quantification of the extravasation of antibodies following lung transplant, and with treatments of IL1β and PBS.

FIGS. 12A-12B show LRA activates the complement system only after extravasation, a process that relies on IL1β secretion. It was observed that LRA induced complement activation specifically after its movement into the extravascular space. In FIG. 12A, left panel, mice were injected with isotype control antibodies (IgG-CT) and subsequently underwent lung transplantation. The lung allografts were then subjected to complement staining using C4D. No signs of complement activation were detected. However, when mice were administered LRA before undergoing lung transplantation, complement deposition became evident (FIG. 12A, right panel). Notably, when IL1r−/− mice which lack receptors for IL1β—received LRA and subsequently underwent lung transplantation, neither complement activation nor LRA extravasation was observed (FIG. 12B)

FIG. 13 shows IL1β is essential for the extravasation of LRA. Normal mice were given LRA and subsequently treated with either PBS, IL1β, or a combination of IL1β and anti-IL1R antibodies. Through immunofluorescence imaging, it was observed that LRA extravasated and settled when IL1β was administered to the mice. In contrast, in mice treated with both IL1 and antibodies targeting its receptors (IL1R), no extravasation was seen.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only and is not intended to be limiting.

### Definitions

The present invention is described herein using several definitions, as set forth below and throughout the application.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus≥10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, “subject” refers to an animal, such as a mammal (including a human), that has been or will be the object of treatment, observation or experiment. “Subject” and “patient” may be used interchangeably, unless otherwise indicated. Mammals include, but are not limited to, mice, rodents, rats, simians, humans, farm animals, dogs, cats, sport animals, and pets. The methods described herein may be useful in human therapy and/or veterinary applications. In some embodiments, the subject is a mammal. In some embodiments, the subject is a human.

As used herein, the terms “administration” and “administering” mean the delivery of a bioactive composition or formulation to a subject by an administration route including, but not limited to, intravenous, intra-arterial, intramuscular, intraperitoneal, subcutaneous, intramuscular, or combinations thereof. In some embodiments, the administration to a subject is intravenous.

Primary Graft Dysfunction

Pre-existing lung restricted autoantibodies (LRA) are associated with a higher incidence of primary graft dysfunction (PGD) although it remains unclear whether LRA can drive its pathogenesis. In syngeneic murine left lung transplant recipients, pre-existing LRA worsened graft dysfunction, which was evident by impaired gas exchange, increased pulmonary edema, and activation of damage-associated pathways in lung epithelial cells. LRA-mediated injury was distinct from ischemia-reperfusion injury since deletion of donor non-classical monocytes as well as host neutrophils could not prevent graft dysfunction in LRA-pretreated recipients. Whole LRA IgG molecule may cause lung injury, which may be mediated by the classical and alternative complement pathways and reversed by complement inhibition. However, deletion of Fc receptors in donor macrophages or mannose-binding lectin proteins failed to rescue lung function. LRA-mediated injury was localized to the transplanted lung and dependent on IL10-mediated permeabilization of pulmonary vascular endothelium which allowed extravasation of antibodies. Genetic deletion or pharmacological inhibition of IL1R in the donor lungs prevented LRA-induced graft injury. In humans, pre-existing LRA was an independent risk factor for severe PGD and could be treated with plasmapheresis and complement blockade. Therefore, pre-existing LRA may compound ischemia-reperfusion injury to worsen PGD for which complement inhibition may be effective.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

Methods

One aspect of the present disclosure includes the use of the existing Luminex test to screen all patients who need lung transplantation, for example Test information: LABScreen autoantibody kits (Cat #LSAUT1-3, One Lambda). The autoantibodies can also be tested using any ELISA or antibody-based assays.

A further aspect of the present disclosure includes the use of anti-IL1β antibodies or anti-IL1 receptor antibodies to treat patients with these autoantibodies to prevent or treat PGD. In some aspects, the anti-IL1β antibodies include Canakinumab, also known by the trade name Ilaris. In some aspects, the anti-IL1 receptor antibodies include Anakinra, also known by the trade name Kineret. In some aspects, the subject is screened for the presence of lung autoantibodies prior to administration of the anti-IL P antibodies or the anti-IL1 receptor antibodies.

A further aspect of the present disclosure includes the use of complement inhibitors against any of the classical and alternate pathway proteins to treat PGD in the presence of autoantibodies. In some aspects, the complement inhibitors include Eculizumab, Cinryze, or Rocunest among others.

In yet another aspect of the present disclosure, a method for treating PGD is provided. The method includes screening the subject for the presence of lung restricted antibodies and administering an anti-IL1β antibody and/or complement inhibitor. In one aspect, the screening step comprises a Luminex test to screen the subject for the lung restricted antibodies.

In yet another aspect of the present disclosure, a method of treating or preventing primary graft dysfunction in a subject is provided. In one aspect, the method includes administering an anti-lung restricted antibody. In one aspect, the anti-lung restricted antibody includes an anti-IL1β antibody. In yet another aspect, wherein the anti-IL1β antibody comprises Canakinumab or Anakinra or derivatives thereof.

In yet another aspect of the present disclosure, a method of treating or preventing primary graft dysfunction in a subject is provided. In one aspect, the method includes administering a complement inhibitor. In one aspect, the complement inhibitor includes Eculizumab, Cinryze, or Rocunest, or derivatives thereof.

In yet another aspect of the present disclosure, a method of determining the likelihood of primary graft dysfunction in a subject is provide. In one aspect, the method includes detecting the presence of chemokine IL1β in a sample from the subject following lung transplantation. In one aspect, the sample is a blood sample, a tissue sample, or mucous sample. In one aspect, the sample is taken from the interstitial space of the lungs.

In yet another aspect of the present disclosure, a method of preventing or reducing the incidence of primary graft dysfunction in a subject is provided. In one aspect, the method includes screening the subject for the presence of lung restricted antibodies and administering an anti-IL1β antibody and/or complement inhibitor to the subject. In one aspect, the administering comprises administering an anti-IL1β antibody. In one aspect, the screening step comprises a Luminex test to screen the subject for the lung restricted antibodies.

In yet another aspect of the present disclosure, a method of treating or preventing primary graft dysfunction in a subject is provided. In one aspect, the complement inhibitor comprises Eculizumab, Cinryze, or Rocunest, or derivatives thereof. In one aspect, the method further comprises administering plasmapheresis and/or a complement blockade. In any aspect of the present disclosure, the subject is a human being.

In yet another aspect of the present disclosure, a method for identifying transplantation patients who are likely to benefit from IL1β therapy through autoantibody testing is provided. In one aspect, a method for selecting patients as candidates for IL1β therapy includes screening a patient to determine whether the patient exhibits autoantibodies against the lungs prior to transplantation. In one aspect, the screening is performed using any autoantibody testing technique, including, but not limited to, Enzyme-Linked Immunosorbent Assay (ELISA), LUMINEX, multiplex immunoassay, or a commercially available test, such as those provided by One Lambda.

In yet another aspect of the present disclosure, a method for targeting IL10 or its receptor IL1R to mitigate the harmful effects of autoantibodies in a patient is provided. In one aspect, the method includes administering to a patient an antibody against IL1β or IL1R, thereby neutralizing the impacts of IL1β.

In yet another aspect of the present disclosure, a method for administering an IL1β-targeting antibody to a patient is provided. In one aspect, the IL1β-targeting antibody is administered at least 30 minutes prior to the reperfusion of the transplanted lungs. In one aspect, administration occurs post skin incision on the recipient at the commencement of the operation. In one aspect, in the case of substantial blood loss (e.g., exceeding 500 cc) during the old lung dissection and before the new lung implantation, at least one additional dose of the IL1β-targeting antibody is administered to the patient.

In one aspect, the IL1β-targeting antibody is a monoclonal or a polyclonal antibody against IL1β. In one aspect, the at least one additional dose is a single dose. In one aspect, more than one dose may be administered. As a non-limiting example, a single dose at 24 hours post-transplantation may be administered to the patient, and again at 48 hours if there is ongoing blood loss following the procedure or if primary graft dysfunction is observed.

In one aspect, IL1R may be employed to counteract IL1β effects. In one aspect, an antibody against IL1R is administered to the to the donor during organ preservation, prior to donor heart cross-clamping. In one aspect, the antibody against IL1R is administered through intravenous injection post skin incision in the donor. In one aspect, the antibody against IL1R is administered as part of the preservative solution during lung perfusion post cross-clamping, or during ex vivo lung perfusion. In one aspect, a second dose of the antibody against IL1R, either intravenously or subcutaneously, may be administered to the recipient within 24 hours for enhanced effectiveness.

### EXAMPLES

### Example 1: Introduction

Primary graft dysfunction (PGD) affects over 50% of recipients following lung transplant and has emerged as the principal risk factor for both short-term mortality as well as long-term graft loss from chronic rejection (1-4). Current empiric therapies to treat PGD are largely ineffective and have the attendant risks of immunosuppression. While ischemia-reperfusion injury remains its predominant cause, the highly variable incidence of PGD suggests additional etiologies for its pathogenesis. A significant percentage of patients undergoing lung transplantation have pre-existing immunoglobulin G (subtype IgG2) autoantibodies against lung-restricted self-antigens collagen type V (COLV) and k-alpha1 tubulin (KAT) (5, 6). The presence of these lung-restricted autoantibodies (LRA) in the recipients is associated with an increased risk of PGD following both human (6) and murine (5, 7-9) lung transplantation. However, the molecular events by which LRA promote PGD and their relationship to transplant-inherent ischemia-reperfusion injury remain unknown. Understanding of the pathogenesis of PGD in recipients with underlying LRA will enable the development of selective therapies to improve post-transplant outcomes.

Pre-existing lung restricted autoantibodies (LRA) are associated with a higher incidence of primary graft dysfunction (PGD) although it remains unclear whether LRA can drive its pathogenesis. Investigators have suggested a mechanism to explain the production of LRA in patients with chronic lung diseases (10). The deletion of self-reactive T lymphocytes against lung self-antigens by the thymus is incomplete and escaped self-reactive T lymphocytes are dynamically suppressed through peripheral regulatory T cells (11). Depletion of regulatory T cells in response to environmental challenge promotes the expansion of self-reactive T lymphocytes and development of LRA (10). The lung self-antigens are non-polymorphic and are normally sequestered from the immune system as they serve as scaffolds for the structural proteins (12). However, during transplantation the self-antigens might be revealed as the structural proteins are cleaved, for example through the activation of matrix metalloproteinases, allowing the pre-existing LRA to bind and promote downstream immune activation (13). These self-antigens are extravascular but the increased vascular permeability that occurs during ischemia-reperfusion injury could allow extravasation of the LRA (14).

In syngeneic murine left lung transplant recipients, pre-existing LRA worsened graft dysfunction, which was evident by impaired gas exchange, increased pulmonary edema, and activation of damage-associated pathways in lung epithelial cells. LRA-mediated injury was distinct from ischemia-reperfusion injury since deletion of donor non-classical monocytes as well as host neutrophils could not prevent graft dysfunction in LRA-pretreated recipients. Whole LRA IgG molecule was correlated with lung injury which was mediated by the classical and alternative complement pathways and reversed by complement inhibition. However, deletion of Fc receptors in donor macrophages or mannose-binding lectin proteins failed to rescue lung function. LRA-mediated injury was localized to the transplanted lung and dependent on IL1β-mediated permeabilization of pulmonary vascular endothelium which allowed extravasation of antibodies. Genetic deletion or pharmacological inhibition of IL1R in the donor lungs prevented LRA-induced graft injury. LRA compounds ischemia-reperfusion injury through the activation of complement resulting in severe PGD. These findings reveal novel pathways through which LRA mediate lung allograft injury (Schematic Figure) that can be targeted clinically. In humans, pre-existing LRA was an independent risk factor for severe PGD and could be treated with plasmapheresis and complement blockade. In conclusion, pre-existing LRA can compound ischemia-reperfusion injury to worsen PGD for which complement inhibition may be effective.

### Example 2: Materials and Methods

Mice and procedures. Male wild type C57BL/6J (B6), Fcrγ−/−, I11r−/−, C1qa−/−, C3^{-/-}, Mbl−/− and Nr4a−/− mice were obtained from Jackson Laboratory. IL-1RA (0.2 ng/g body weight intravenously (i.v.), Sigma) was used for IL1β-IL1R antagonism. C1INH (0.4U/g body weight, Berinert®, CSL Behring) and LNP023 (Factor B inhibitor, 30 μg/g body weight, Adooq Bioscience) were injected via i.v. in recipients 24 hours before and 1 hour after lung transplant. IL1β (10 μg/kg; i.v.; ThermoFisher) was injected 8 hours prior LRA injection. Neutrophils were depleted by using Ly6G antibody (12.5 mg/Kg body weight, clone 1A8, bio X Cell). Complement C5 was inhibited by using a C5 blocking antibody (100 μg/mouse, 1 hour before LTx; kindly provided by Alexion Pharmaceuticals). Control mice were treated with the same amounts of IgG isotype control antibodies (bio X Cell). All mice were maintained in a specific pathogen-free facility at the Center for Comparative Medicine at Northwestern University and used for the described experiments at the age of 9-14 weeks and between 24-28 g of body weight.

### Mouse Lung Transplant.

Orthotropic murine left lung transplantation was performed as previously described (44). Briefly, donor mouse was anesthetized with a mixture of xylazine (10 mg/kg) and ketamine (100 mg/kg). Donor lungs were flushed through the pulmonary artery with 3 ml of saline solution and the heart-lung block was excised and kept in cooled (4° C.) preservative solution. The bronchus, pulmonary vein, and artery were dissected and prepared for anastomosis. A customized cuff made of a Teflon intravenous catheter was applied to the vascular structures and fixated with a 10-0 nylon ligature. After placement of a micro vessel clip on the bronchus to avoid airway infiltration with preservative solution, the graft was stored at 4° C. for a period of 90-120 minutes of cold ischemic time prior to implantation. The recipient mouse received subcutaneous buprenorphine (0.1 mg/kg) 30 min prior to the thoracic surgical incision and every 6 h as needed after the transplant procedure. The recipient mouse was intubated and a left-sided thoracotomy was performed within the third intercostal space. The recipient's native lung was gently clamped and pulled out of the thoracic cavity. The space between the artery, the vein and the bronchus was dissected separately. The artery and vein were temporarily occluded using 8-0 nylon ligatures. The anastomoses were completed by fixating each cuff with 10-0 nylon ligatures. The 8-0 ligatures were released (first vein, then artery) and the lung inflated. The chest incision was closed and recipients separated from the ventilator when spontaneous respiration resumed. No antibiotics or immunosuppressive agents were used postoperatively in any group. LRA were administered intravenous in recipient mouse: 150 μg each (anti-Col V and anti-KAT) or 300 ug isotype rabbit IgG (ThermoFisher Scientific) 24 hours before and 1 hour after lung transplantation.

Antibodies to KAT and Col-V. Rabbit polyclonal IgG antibodies to KAT and Col-V were produced against KAT and Col-V proteins as previously described (45). Purified antibodies were endotoxin free by limulus amebocyte lysate assay. F(ab′)₂ fragments of IgG antibodies to KAT and Col-V were prepared with Pierce™ F(ab′)₂ Preparation Kit as described by the manufacturer (ThermoFisher Scientific). For two photon microscopy, anti-Col-V antibody was conjugated to R-PE using PE/R-Phycoerythrin Conjugation Kit-Lightning-Link® (ab102918, Abcam).

Arterial blood gases. Arterial blood gases were measured using a fraction of inspired oxygen of 100% after the right pulmonary hilum was clamped for 5 minutes (8). Blood was collected by left ventricular puncture.

Weight to Dry Ratio. The transplanted left lung was harvested after reperfusion at defined time points, weighed, and then placed at 54° C. until a stable dry weight was achieved. The ratio of wet weight to dry weight was then calculated as an indicator of pulmonary edema.

Flow cytometry. Mouse lung was digested, and single cell suspensions were prepared as previously described (44). Cell suspensions underwent red blood cell lysis using Pharm Lyse buffer (BD Biosciences). Live/dead staining was performed in protein-free solution (HBSS) using fixable viability dye eFluor 506 (eBioscience), followed by incubation with FcR-blocking reagent (Miltenyi Biotec). Antibodies utilized for murine cell staining included rat anti-mouse CD45-FITC (30-F11, BioLegend), rat anti-mouse Ly6C-eFluor450 (HK1.4, eBiosciences), rat anti-mouse I-A/I-E-PerCPCy5.5 (M5/114.15.2, BioLegend), rat anti-mouse CD45-APC (30-F11, BioLegend), rat anti-mouse Ly6G-AlexaFluor 700 (1A8, BioLegend), rat anti-mouse NK1.1-AlexaFluor 700 (PK136, BD), rat anti-mouse CD11b-APCCy7 (M1/70, BioLegend), rat anti-mouse CD64-PE (X54-5/7.1, BioLegend), rat anti-mouse SiglecF-PECF594 (E50-2440, BD), rat anti-mouse CD11c-PECy7 (HL3, BD). For neutrophil quantification, 123Count eBeads (Invitrogen) were added. Flow analysis of fixed samples was done on a BD FACS Symphony A5-Laser Analyzer at the Northwestern University Robert H. Lurie Comprehensive Cancer Center Flow Cytometry Core facility. Acquired data was analyzed with FlowJo v10.8 (FlowJo).

Histology. Tissue sections were stained with hematoxylin and eosin and analyzed blindly. Images were obtained on a Nikon Eclipse microscope (Nikon,Minato-ku, Tokyo) or Olympus DP-71 (Olympus, Center Valley, PA), and morphometric analysis performed using Nikon Elements software. Analysis was performed on 10 different areas of the lungs, and at least 10 high-powered fields were analyzed in each area.

Single-cell RNAseq. Single-cell suspensions from 24 hours post-transplant allografts from isotype or LRA treated mice were prepared as described before (18). Allograft was removed and digested with 3 mL dispase (Corning) with DNase I (Sigma), and gently teased using forceps into small (1-2 mm) fragments followed by incubation at room temperature with gentle agitation for 30 minutes. The resulting suspension (in DMEM+5% FBS) was passed through 70 μm cell strainer, erythrocytes were lysed and filtered through 40 m cell strainers. Cells were counted using AO/PI and Cellometer K2 (Nexcelom), and cell viability exceeded 90%. Single-cell 3′ RNA-Seq libraries were prepared using Chromium Single Cell V2 Reagent Kit and Controller (10× Genomics). Libraries were assessed for quality (TapeStation 4200, Agilent) and then sequenced on NextSeq 500 or HiSeq 4000 instruments (Illumina). Initial data processing was performed using the Cell Ranger version.

6.0 pipeline (10× Genomics), reads were mapped to mm10 version of the mouse genome, Ensemble build 105. Ambient RNA contamination was removed by SoupX package (46). Doublets were evaluated by scrublet and removed (47). Downstream single cell RNA-seq analysis was performed using Seurat Package version 4.0 following the standard workflow posted on Satija lab website (https://satijalab.org/seurat/) (48). Cells with unique feature counts less than 200 or over 7500 were removed. Cells with RNA counts less than 400 or over 40000 were removed. Cells having larger than 10% mitochondrial counts were removed. The data was normalized by “LogNormalize” method and variable features were selected by “vst” method. Reciprocal principal component analysis (RPCA) was used for the data integration. Cell types were identified using both manual annotations based on positive markers from FindAllMarkers function in the Seurat pipeline and a supervised annotation tool, SingleR (49). The RNA-seq data can be found in the GEO repository #GSE211501.

Two-photon intravital lung microscopy. Two-photon intravital lung microscopy imaging was performed using a Nikon A1R-MP+ multiphoton microscope system with a Coherent Chameleon Vision titanium sapphire laser. As previously described (18), mice were anesthetized with an i.p. injection of ketamine (80 mg/kg) and xylazine (10 mg/kg), intubated orotracheally and ventilated with room air at a rate of 120 breaths/minute with a tidal volume of 0.5 mL. A Left thoracotomy was performed to expose the left lung, and the lung was imaged using a custom-built chamber maintained at 37° C. Vetbond was used to attach the lung tissue to the bottom of the cover glass without direct pressure on the exposed lung. For time-lapse imaging of location and extent of anti-ColV-PE deposition in lungs, 37 video-rate frames (0.5 seconds per slice) were averaged during the acquisition to match the ventilator rate and minimize movement artifacts. To visualize blood vessels, 25 μL of FITC-dextran (2M kDa, 5 mg/mL, Thermo Fisher Scientific) in 25 μL of PBS was injected i.v. 5 minutes prior to imaging. For each mouse, images of 512×512 m in the x and y dimensions were acquired for 5 minutes prior to and for 15 minutes following injection of anti-ColV-PE (1 mg/kg i.v.) using a water immersion lens (Apo LWD 25×1.10 W DIC N2) at an excitation wavelength tuned at 1000 nm. Images were processed and analyzed using Nikon NIS-Elements NIS.ai and Bitplane Imaris software for background/fluorophore spillover subtraction and fluorescence quantification of deposited fluorophores. Data were transferred and plotted in Graph Pad Prism 9.0 (Sun Microsystems) for analysis and creation of graphs.

Broncho alveolar lavage fluid (BALF). Right pulmonary hilum was clamped, and left lung BALF was obtained by instilling lung airways 2 times with 0.5 mL PBS. BALF was centrifuged and the supernatant was used for complement C3 determination.

ELISA. Mouse complement C3 ELISA were performed using commercially available kits accordingly to the Manufacturer's instructions (Abcam). To determine autoantibodies in patients' serum, LABScreen autoantibody kits (Cat #LSAUT1-3, One Lambda) was used according to manufacturer's instructions.

Immunohistochemistry. Mice were anesthetized with pentobarbital sodium (50 mg/kg, i.p.), lung tissues were excised, and fixed with 10% Neutral buffered formalin. Paraffin-embedded sections were incubated with rabbit anti-C4d IgG (1:200, overnight at 4° C., Hycult biotech, #HP8033) or rabbit anti-C5b-9 IgG (Bioss antibodies, #bs-2673R) and then successively reacted with Donkey Anti-Rabbit IgG H&L (Alexa Fluor® 488) (1:500, 1 h at RT, Abcam, #ab150073). For LRA deposition, paraffin-embedded sections were incubated directly with Donkey Anti-Rabbit IgG H&L (Alexa Fluor® 488) as above. Nuclei were stained with Hoechst 33342 (Millipore Sigma). Confocal images were acquired using a Zeiss Axio Imager Z2 with ApoTome.2 microscope equipped with Axiocam 503 Mono, X-Cite 120 LED Boost System, and Zen 2.3 software (Carl Zeiss).

In vitro endothelial cell permeability. Lung primary Lung microvascular endothelial cells (Cell Biologics #C57-6011) were grown in endothelial cell medium phenol red free (ScienCell), seeded in collagen coated inserts and permeability was determined using a vascular permeability assay kit according to manufacturer instructions (Millipore). Fluorescein isothiocyanate (FITC)-conjugated anti-Col-V antibody (labelled using FITC Conjugation Kit (Fast)—Lightning-Link®, Abcam) was added on well in the absence or presence of 50 ng/ml recombinant mouse IL10 (Thermo Scientific) and fluorescence was red with a plate reader with filters appropriate for 485 nm and 535 nm excitation and emission, respectively.

Definition of primary graft dysfunction. Patients with no evidence of pulmonary edema on chest X-ray are considered grade 0. Absence of invasive mechanical ventilation was graded according to the PaO₂/FIO₂ ratio, using methods similar to those receiving mechanical ventilation. If PaO₂ is not available for calculation of a PaO₂/FiO₂ ratio, then an oxygen saturation/FiO₂ ratio was used to calculate. Grade3; PaO₂:FiO₂ ratio is <200. The worst PaO₂/FiO₂ ratio within 72 hours after lung transplant was used. Use of extracorporeal lung support (ECLS) with bilateral pulmonary edema on chest X-ray image was graded as grade 3.

Immunosuppressive therapy. All lung transplant recipients received standardized Immunosuppressive therapy. Methylprednisolone 500 mg IV intraoperatively and Basiliximab 20 mg IV intraoperatively and post-operative day (POD) 4 were given as induction immunosuppressive therapy.

Maintenance Immunosuppression was given as followed: Prednisone 0.5 mg/kg PO daily from POD 1, Mycophenolate mofetil 1000 mg IV BID from POD 1, and Tacrolimus 0.015 mg/kg total daily dose sublingual, targeting from 8 to 12 ng/ml.

Patients with LRA that developed PGD underwent treatment for antibody mediated rejection using intravenous methylprednisolone (1000 mg) for 3 days, daily plasmapheresis for 3 days followed by intravenous immunoglobulin (1 mg/kg) and eculizumab (1200 mg, 900 mg, and 600 mg on day 1, 2, and 3).

Statistical Analysis.

### Murine

Mouse data analysis was performed using Prism 8 (GraphPad Software). Results are expressed as mean±SD and the n values for each data set are provided in the figure legends. Statistical significance was assessed by 2-tailed Student's t test, or one-way ANOVA followed by Tukey's post-hoc test. A p value less than 0.05 was considered significant.

### Human

Incidence of primary graft dysfunction grade 3 after lung transplant was compared between lung restricted antibodies positive group and lung restricted antibodies negative group. Continuous variables were compared using t-test and reported as means±SD. Categorical variables were compared using Fisher exact test and reported as number (percentage). P-values <0.05 were accepted as statistically significant. Logistic regression model was used to derive odds ratios and 95% confidence intervals. To build these models, a univariate analysis was performed and included all predictors if the test had a p-value of 0.2 or less. To assess the overall goodness of fit, Gronnesby and Borgan tests were used. Statistical analyses were performed using EZR (Saitama Medical Center, Jichi Medical University, Saitama, Japan), which is a graphical user interface for R (The R Foundation for Statistical Computing, Vienna, Austria). It is a modified version of R commander designed to add statistical functions frequently used in biostatistics.

### Example 3: Intact LRA Promote Primary Graft Dysfunction

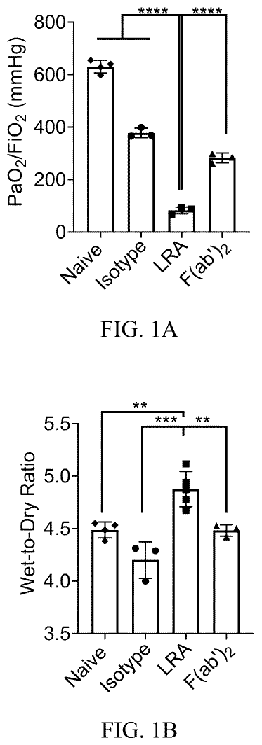

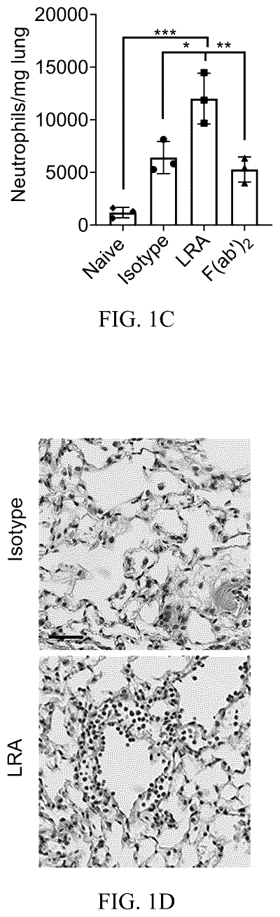

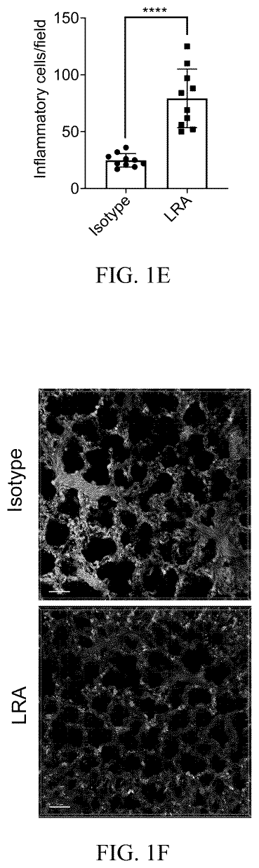

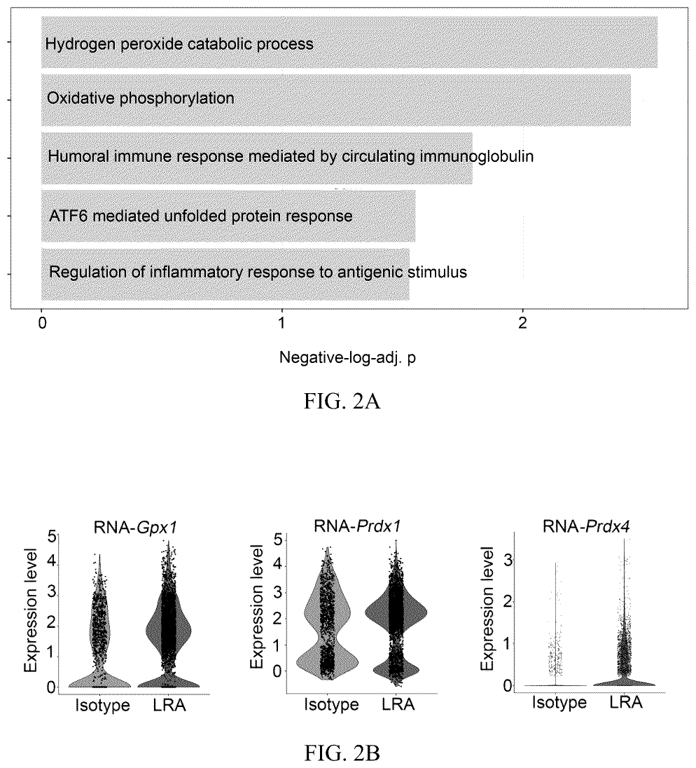

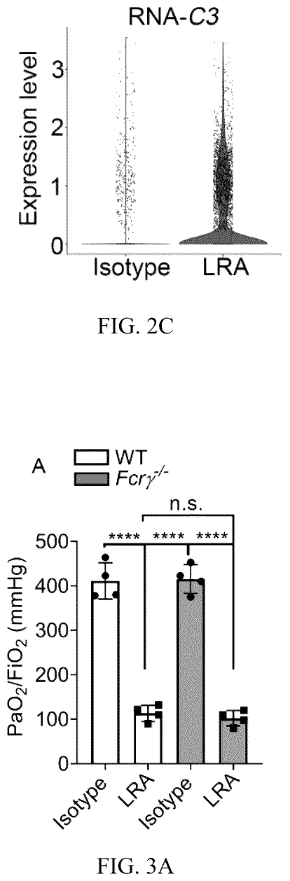

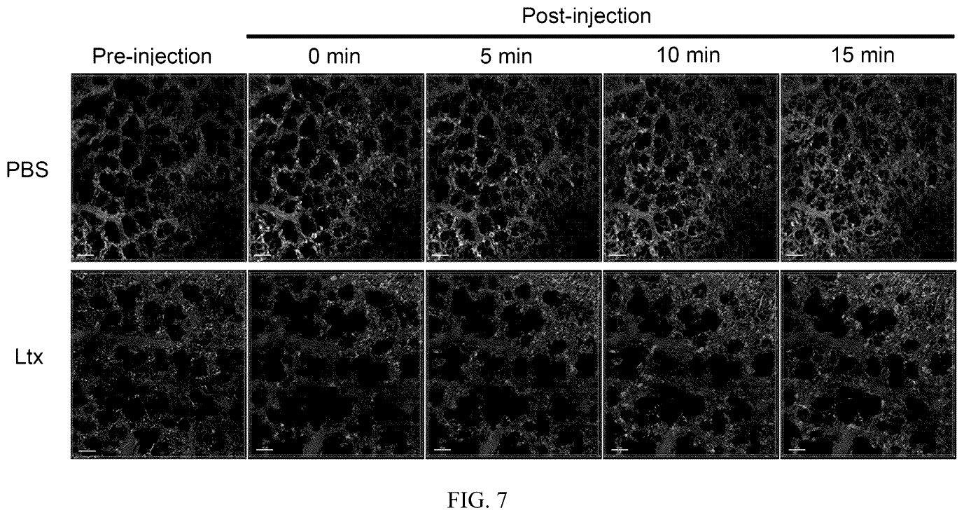

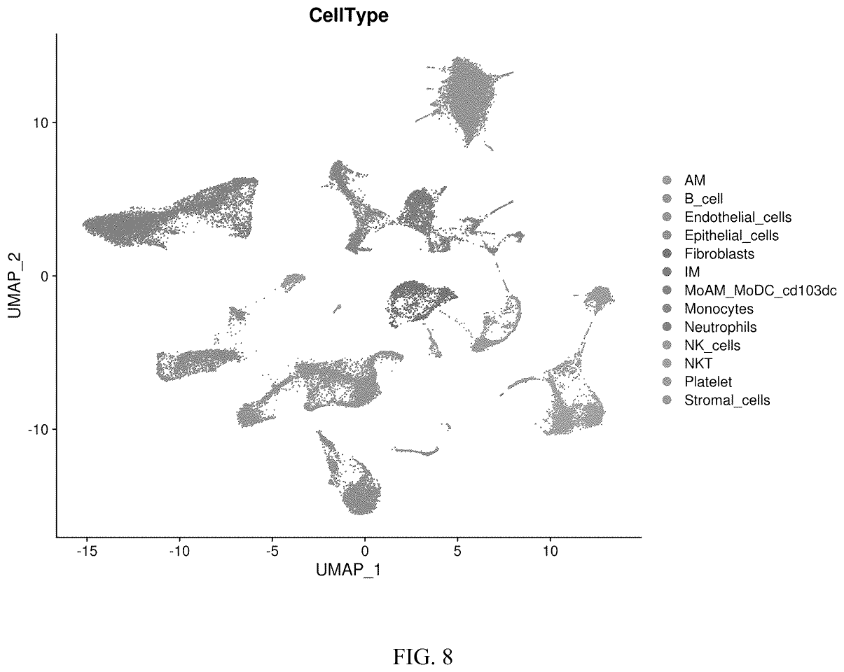

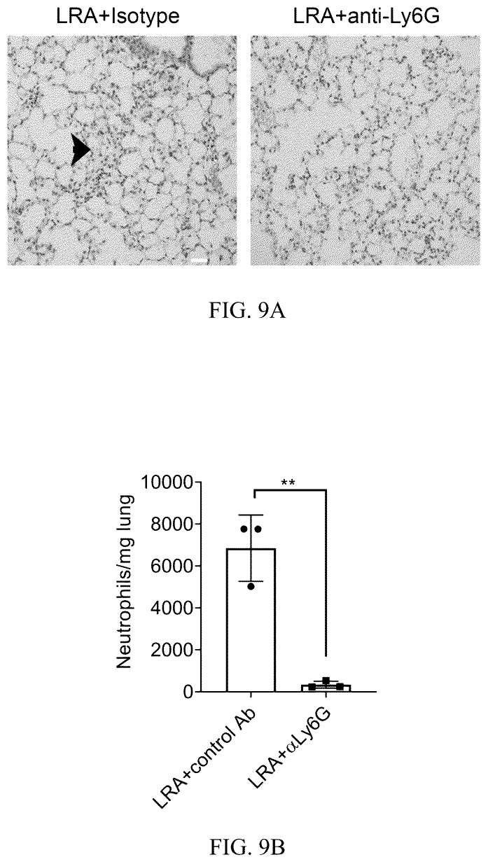

LRA belong to the IgG family of immunoglobulins (5, 6) which are comprised of Fc and F(ab′)₂ fragments. The F(ab′)₂ fragment exhibits sequence variability and recognizes antigens, while the Fc fragment provides interaction sites for effector molecules such as complement and Fcγ receptors (FcγR) (15). However, F(ab′)₂ can also independently interact with FcγR to mediate immunological effects (16). To determine the pathogenicity of LRA, IgG isotype, LRA, or the cleaved F(ab′)₂ fragment was injected into recipient mice prior to transplantation. As expected, ischemia-reperfusion injury was inherent to this model and experienced by all animals, evident by decreased lung graft function in the recipients of IgG isotype control compared to naive lung (Isotype PaO₂:FiO₂ ratio: 452.3±63 mmHg, Naïve PaO₂:FiO₂ ratio: 621.9±32 mmHg; p<0.0001). Recipients injected with whole LRA IgG molecule showed significant worsening in lung graft function (PaO₂:FiO₂ ratio: 104.2±41 mmHg; FIG. 1A) as well as increased pulmonary edema (FIG. 1B) while those injected with F(ab′)₂ fragments demonstrated expected levels of lung graft dysfunction resulting from ischemia-reperfusion injury (FIG. 1A and FIG. 1i). There was increased neutrophil infiltration determined by flow cytometry in mice with whole LRA molecule (FIG. 1C) along with capillaritis and inflammatory cells found on histological analysis (FIG. 1D and FIG. 1E). In addition, two photon imaging (FIG. 1F, FIG. 7) showed increased LRA deposition in the allograft after lung transplantation. Unbiased transcriptomic analysis using single cell RNA sequencing (FIG. 8) revealed activation of several damage-associated pathways (FIG. 2A), including oxidative stress (reflected as increased expression of anti-oxidant proteins (FIG. 2B)), and complement (increased C3 expression, FIG. 2C) in the epithelial cells during LRA-mediated lung graft injury.

### Example 4: LRA Activate Classical and Alternative Complement Pathways to Mediate Lung Graft Injury

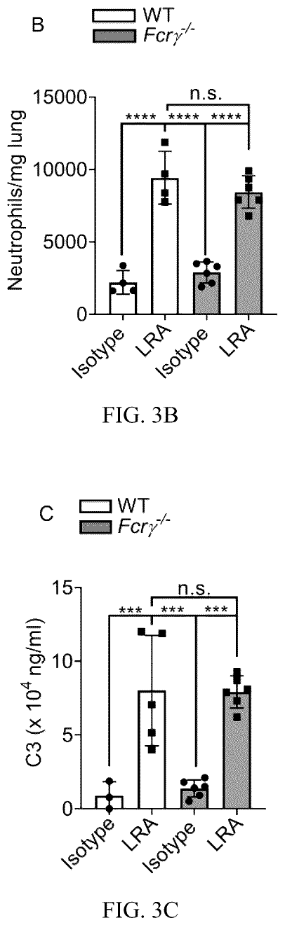

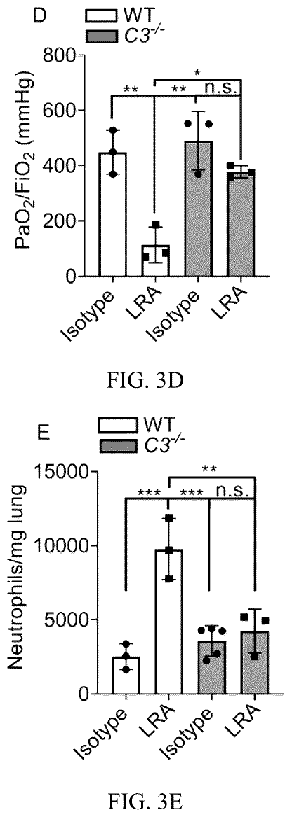

Since F(ab′)₂ could not induce PGD, the Fc region was reasoned to be correlated with the pathogenic effects of LRA. Accordingly, whether LRA-mediated lung injury occurred through FcγR or complement activation (17) was determined. Donor lungs from Fcrγ−/− mice transplanted into syngeneic recipients pre-treated with LRA experienced severe lung injury, similar to those from wild-type donor lungs (FIG. 3A and FIG. 3B). These mice were used as donors and not recipients as the predominant macrophages in the donor lungs at 24 hours are of donor origin (18). This led to a further hypothesis that LRA activates complement to induce lung graft dysfunction.

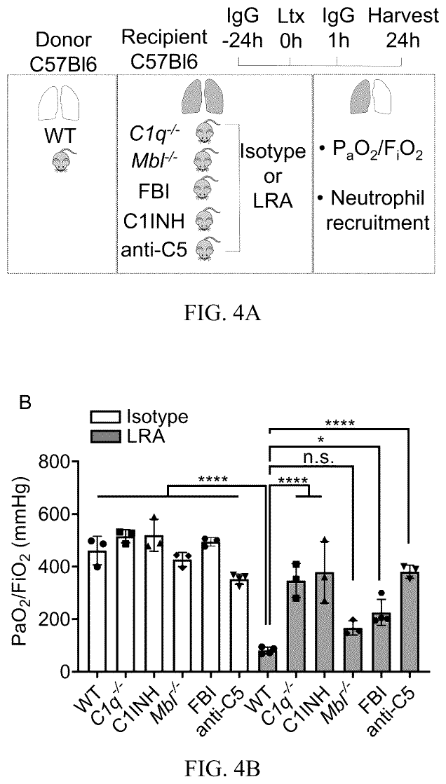

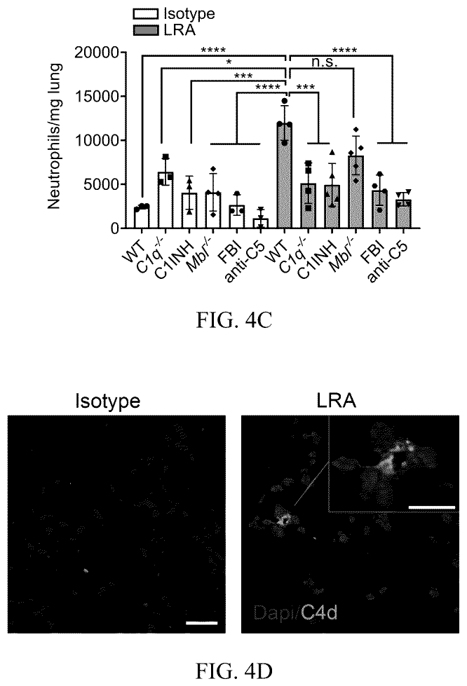

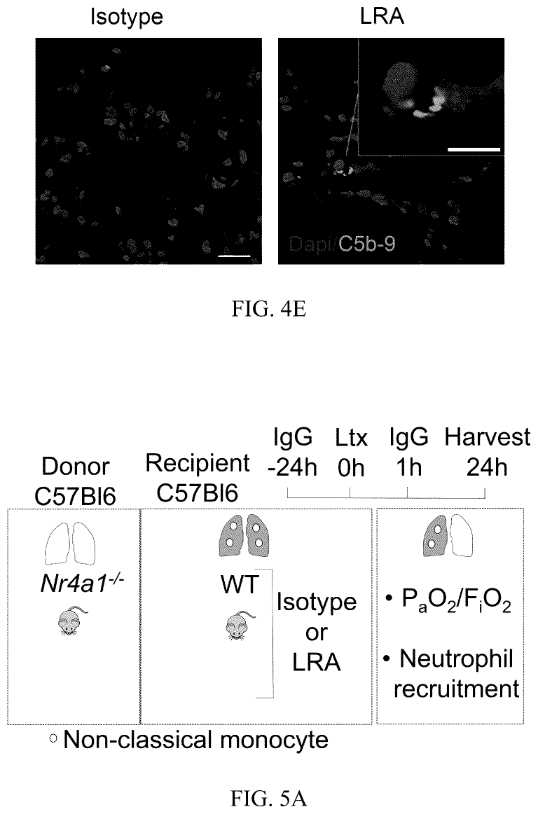

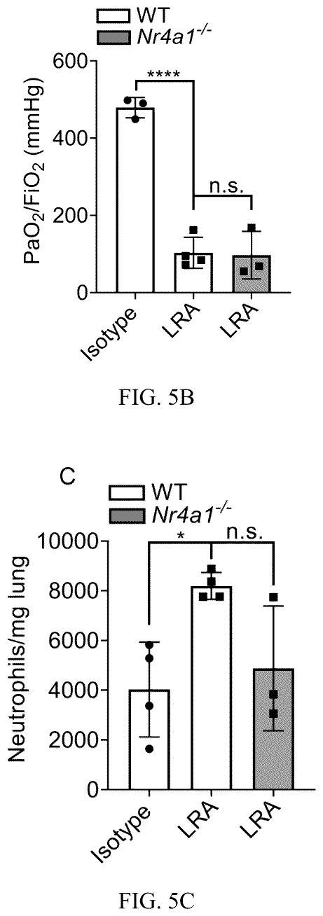

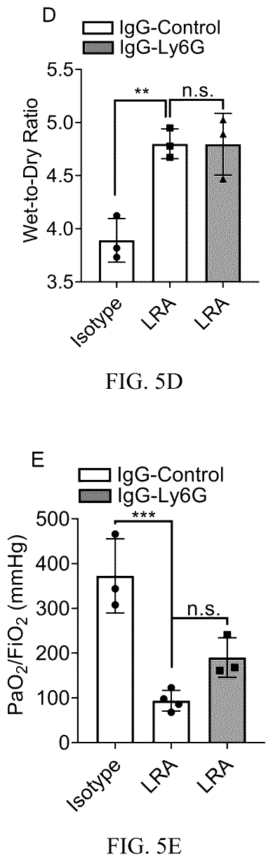

Component C3 was studied as it acts as a point of convergence for the different complement pathways. Indeed, C3 levels were elevated in the BAL of grafts from wild-type or Fcrγ−/−recipients pre-treated with LRA but not in those that received isotype control (FIG. 3C). Moreover, genetic deletion of C3 in recipient mice resulted in near complete protection against LRA-induced lung graft dysfunction (FIGS. 3D and 3E). Next, the specific complement pathway activated by LRA was determined using genetic deletion and pharmacological inhibition. Wild-type donor lungs were transplanted into C1q−/−(classical pathway), Mbl−/−(mannose binding lectin, lectin pathway), or FBI (Factor B inhibitor, alternative pathway)-treated, recipients. Each of these recipients also received LRA or isotype control antibodies prior to the transplant. Pre-existing LRA induced severe lung graft dysfunction in Mbl−/− mice while the C1q−/− and FBI-treated recipients were protected (FIGS. 4B and 4C). Furthermore, treatment of recipient mice with a pharmacological C1 inhibitor (C1INH) resulted in protection of the transplanted lungs from LRA-induced injury as well as associated increase in neutrophil recruitment (FIGS. 4B and 4C). Immunohistochemistry confirmed complement C4d deposition, a marker of classical pathway activation, in the lung grafts of LRA-treated recipients (FIG. 4D). All three complement pathways can induce the formation of the membrane attack complex (MAC). MAC assembly requires the sequential and irreversible association of complement proteins C5b, C6, C7, C8 and C9 (19, 20). Immunohistochemical analysis showed MAC deposition in grafts from LRA recipient mice (FIG. 4E). Inhibiting activation of C5, and thus the generation of C5a and MAC, has shown great therapeutic benefit in complement-driven inflammatory diseases (21). Hence, C5 was inhibited by using a C5 blocking antibody and found prevention of LRA-induced graft dysfunction (FIGS. 4B and 4C). Together, these data suggested that LRA activates both the classical and alternative complement pathways to mediate lung graft injury.

### Example 5: IL1p is Necessary for Vascular Endothelial Permeabilization and Extravasation of LRA

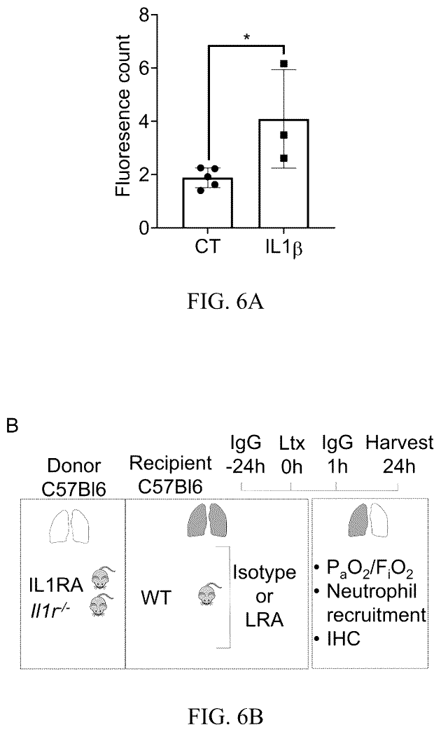

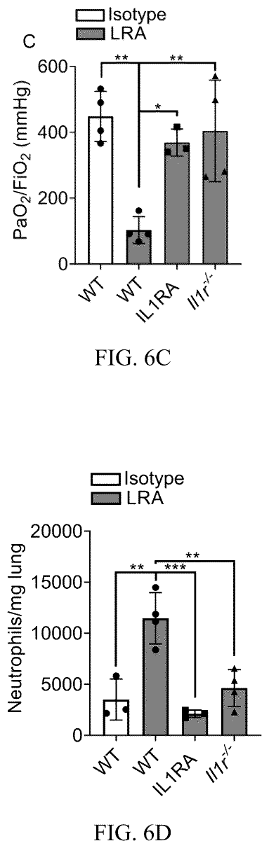

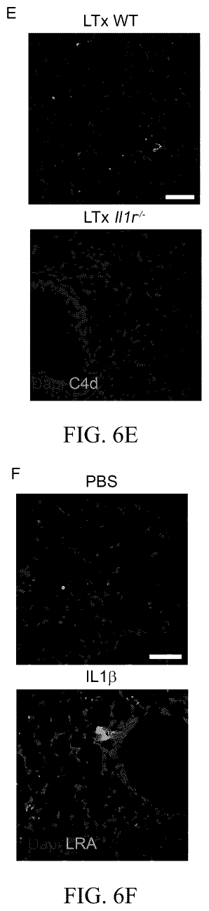

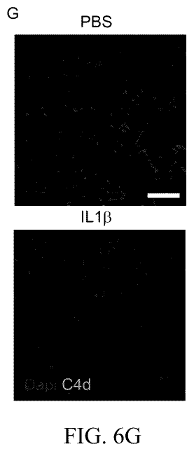

The native lung of the LRA-treated recipients showed no histological evidence of injury. Additionally, there was no increase in neutrophils in the native lungs of LRA-treated recipients (Neutrophils/mg lung: Isotype: 1685±760; LRA: 1445±1008). Lung-restricted antigens are present in the interstitial space (12) and, hence, it was next hypothesized that extravasation of LRA into the interstitial space, that might uniquely occur in the transplanted lung graft, was necessary for the activation of complement. It was previously shown that IL1β can increase pulmonary vascular permeability and allow cellular extravasation (24). Hence, whether release of IL1β within the transplanted lung was necessary to permit extravasation of circulating LRA and gain access to the antigens was tested. Primary pulmonary endothelial cells were first treated in vitro with exogenous IL1β (50 ng/ml) and found that it significantly increased endothelial permeability and the passage of LRA through the barrier (FIG. 6A). Then wild-type, IL1R antagonist (IL1RA)-treated, or I11r−/−lungs were transplanted into wild-type LRA-treated syngeneic recipients. Donor lungs treated with IL1R antagonist or those from I11r−/− mice showed preserved lung graft function (FIG. 6C), decreased neutrophil influx (FIG. 6D) and no C4d deposition (FIG. 6E). There was no difference in graft function among the isotype and LRA group treated with IL1RA or when I11r−/−donor lung was used (PaO₂/FiO₂ (mmHg): IL1RA-isotype: 515.7±24 (n=3); I11r−/−-isotype: 412±141 (n=3); IL1RA-LRA: 368±41 (n=3); I11r−/−-LRA: 404±154 (n=4)). To further investigate the role of IL1P, we injected wild-type mice with IL1β or PBS, 8 hours prior to injecting LRA, and determined LRA deposition and complement activation. Retention of LRA was found (FIG. 6F) with IL1β pre-treatment in naive lungs, similar to lung allografts (FIG. 7) but it was not associated with C4d deposition (FIG. 6G), indicating that IL1β is necessary for the extravasation of LRA but is not sufficient to trigger LRA-mediated complement activation.

### Example 6: Pre-Existing LRA is an Independent Risk of Human PGD

LRA in 56 patients was analyzed undergoing lung transplantation. The cumulative prevalence of detectable pre-existing collagen type V, and k-alpha 1 tubulin antibodies was 14.3% in this cohort. The demographic profile of the patients is presented in Table 1.

TABLE 1
Characteristics of Patients in study cohort

| | Overall | | LRA | Non LRA | | |
|---|---|---|---|---|---|---|
| Variable | (n = 56) | | (n = 8) | (n = 48) | | P value |
| Age, years | 59.5 ± 10.7 | | 61.0 ± 8.6 | 59.3 ± 11.0 | | 0.69 |
| Female | 21 | (37.5%) | 2 (25.0%) | 19 | (43.1%) | 0.69 |
| BMI, kg/m2 | 25.8 ± 3.6 | | 27.3 ± 4.2 | 25.2 ± 3.4 | | 0.18 |
| BSA, m2 | 1.8 ± 0.2 | | 1.8 ± 0.1 | 1.8 ± 0.2 | | 0.81 |
| Smoking history | 33 | (58.9%) | 4 (50.0%) | 29 | (65.9%) | 0.90 |
| Hypertension | 29 | (51.7%) | 4 (50.0%) | 25 | (56.8%) | 0.82 |
| Diabetes | 19 | (33.9%) | 2 (25.0%) | 17 | (38.6%) | 0.71 |
| CKD | 6 | (10.7%) | 1 (12.5%) | 5 | (11.3%) | 0.91 |
| Panel Reactive Antibodies | 12 | (21.4%) | 3 (37.5%) | 9 | (20.4%) | 0.34 |
| Donor Specific Antibodies | 2 | (3.5%) | 1 (12.5%) | 1 | (2.7%) | 0.26 |
| Laboratory | | | | | | |
| Hemoglobin, g/dL | 11.9 ± 2.8 | | 13.5 ± 3.0 | 11.6 ± 2.6 | | 0.07 |
| WBC, 1,000/mm3 | 10.6 ± 4.7 | | 11.9 ± 5.0 | 10.4 ± 4.6 | | 0.41 |
| Platelets, 1,000/mm3 | 223.7 ± 75.4 | | 252.1 ± 81.9 | 218.8 ± 71.3 | | 0.25 |
| Sodium, mEq/L | 140.2 ± 2.2 | | 140.0 ± 2.1 | 140.3 ± 2.2 | | 0.73 |
| BUN, mg/dL | 16.0 ± 5.1 | | 14.8 ± 3.0 | 16.1 ± 5.3 | | 0.51 |
| Creatinine, mg/dL | 0.7 ± 0.1 | | 0.8 ± 0.1 | 0.7 ± 0.1 | | 0.32 |
| ALT, U/L | 19.5 ± 15.4 | | 24.3 ± 28.0 | 18.7 ± 11.7 | | 0.35 |
| AST, U/L | 30.7 ± 20.3 | | 33.0 ± 35.1 | 30.3 ± 16.4 | | 0.73 |
| Albumin, g/dL | 5.1 ± 8.5 | | 5.9 ± 2.8 | 3.9 ± 0.4 | | 0.01 |
| Total bilirubin, mg/dL | 0.7 ± 0.6 | | 0.7 ± 0.5 | 0.7 ± 0.6 | | 0.96 |
| INR | 1.1 ± 0.2 | | 1.1 ± 0.2 | 1.1 ± 0.2 | | 0.92 |
| ABG (at cannulation) | | | | | | |
| pH | 7.3 ± 0.1 | | 7.3 ± 0.1 | 7.3 ± 0.1 | | 0.42 |
| PaCO2 | 49.6 ± 12.0 | | 43.7 ± 10.0 | 51.0 ± 12.0 | | 0.11 |
| PaO2 | 301.6 ± 108.3 | | 306.2 ± 106.1 | 300.0 ± 109.0 | | 0.90 |
| HCO3 | 28.0 ± 5.4 | | 25.3 ± 3.7 | 28.5 ± 5.3 | | 0.11 |
| Donor | | | | | | |
| Age, years | 34.5 ± 11.5 | | 34.8 ± 11.5 | 34.4 ± 11.5 | | 0.93 |
| Female | 23 | (41.0%) | 2 (25.0%) | 21 | (47.7%) | 0.45 |
| Cause of death | | | | | | |
| Head trauma | 19 | (33.9%) | 4 (50.0%) | 15 | (31.2%) | 0.42 |
| Drug overdose | 19 | (33.9%) | 2 (25.0%) | 17 | (35.4%) | 0.70 |
| Other | 18 | (32.1%) | 2 (25.0%) | 16 | (33.3%) | 0.92 |

Continuous data are shown as means ± standard deviation (SD).
BMI, body mass index;
BSA, body surface area;
CKD, chronic kidney disease;
WBC, white blood cell;
BUN, blood urea nitrogen;
AST, aspartate aminotransferase;
ALT, Alanine aminotransferase;
INR, international normalized ratio

In the study cohort, 4 (7.1%) patients developed PGD grade 3; two (25%) with and two (4.5%) without pre-existing LRA (p=0.09, Table 2).

TABLE 2
Post-operative outcomes in the LRA and non-LRA cohorts

| | Overall | LRA | Non LRA | P |
|---|---|---|---|---|
| Variable | (n = 56) | (n = 8) | (n = 48) | value |
| PGD grade 3 | 4 (7.1%) | 2 (25.0%) | 2 (4.4%) | 0.09 |
| Ventilator use (days) | 4.7 ± 7.3 | 3.8 ± 3.7 | 4.9 ± 9.9 | 0.77 |
| ICU stay (days) | 11.7 ± 11.0 | 9.2 ± 3.6 | 11.7 ± 11.8 | 0.54 |
| Hospital stay (days) | 21.6 ± 23.8 | 27.1 ± 40.5 | 20.7 ± 19.3 | 0.49 |

PGD, primary graft dysfunction;
ICU, intensive care unit

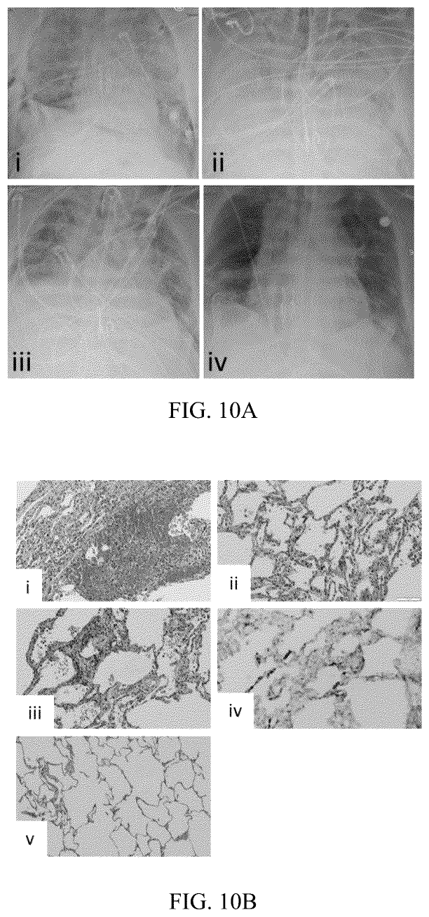

Logistic regression model revealed that only pre-existing LRA was an independent predictor of PGD grade 3 after lung transplant (Table 3 and Table 4). In patients with pre-existing LRA, a post-reperfusion biopsy was performed at 60 minutes. Both patients with pre-existing LRA who developed PGD grade 3 revealed histological features reminiscent of acute antibody mediated rejection with complement (C4d) deposition (FIG. 10). These patients were treated with the complement inhibitor eculizumab, along with plasma exchange, and experienced resolution of lung allograft dysfunction (FIG. 10).

TABLE 3
Univariate Logistic Regression Analysis: Predictors
of Postoperative Primary Graft Dysfunction

| Variable | OR | P value | 95% Cl |
|---|---|---|---|
| LRA | 7.33 | 0.06 | 0.87-62.2 |
| Age, years | 0.50 | 0.56 | 0.04-5.15 |
| Female | 0.45 | 0.49 | 0.04-4.35 |
| BMI, kg/m2 | 1.14 | 0.38 | 0.84-1.54 |
| BSA, m2 | 6.55 | 0.42 | 0.60-6.49 |
| Smoking history | 2.00 | 0.56 | 0.19-20.6 |
| Hypertension | 0.85 | 0.87 | 0.11-6.53 |
| Diabetes | 1.94 | 0.52 | 0.25-1.50 |
| CKD | 3.00 | 0.37 | 0.26-3.46 |
| Panel Reactive Antibodies | 1.47 | 0.28 | 0.58-2.73 |
| Donor Specific Antibodies | 1.22 | 0.72 | 0.46-1.62 |
| Laboratory | | | |
| Hemoglobin, g/dL | 0.98 | 0.72 | 0.65-1.34 |
| WBC, 1,000/mm3 | 0.93 | 0.70 | 0.73-1.21 |
| Platelets, 1,000/mm3 | 0.98 | 0.14 | 0.97-1.01 |
| Sodium, mEq/L | 0.94 | 0.81 | 0.61-1.25 |
| BUN, mg/dL | 0.88 | 0.33 | 0.67-1.14 |
| Creatinine, mg/dL | 3.01 | 0.40 | 0.04-8.66 |
| ALT, U/L | 0.98 | 0.70 | 0.89-1.07 |
| AST, U/L | 1.01 | 0.56 | 0.97-1.05 |
| Albumin, g/dL | 1.12 | 0.43 | 0.84-1.49 |
| Total bilirubin, mg/dL | 2.23 | 0.17 | 0.71-7.00 |
| INR | 0.73 | 0.89 | 0.01-5.93 |
| ABG (at cannulation) | | | |
| pH | 1.53 | 0.73 | 0.87-1.52 |
| PaCO2 | 1.04 | 0.22 | 0.97-1.12 |
| PaO2 | 0.99 | 0.60 | 0.98-1.00 |
| HCO3 | 1.09 | 0.30 | 0.92-1.28 |
| Donor | | | |
| Age, years | 1.01 | 0.76 | 0.92-1.11 |
| Female | 0.42 | 0.47 | 0.04-4.36 |
| Cause of death | | | |
| Head trauma | 1.30 | 0.59 | 0.87-1.68 |
| Drug overdose | 1.06 | 0.98 | 0.78-1.34 |
| Other | 1.24 | 0.76 | 0.89-1.29 |

LRA, lung restricted antibody;
BMI, body mass index;
BSA, body surface area;
CKD, chronic kidney disease;
WBC, white blood cell;
BUN, blood urea nitrogen;
AST, aspartate aminotransferase;
ALT, Alanine aminotransferase;
INR, international normalized ratio

TABLE 4
Multivariate Logistic Regression Analysis: Predictors
of Postoperative Primary Graft Dysfunction

| Variable | OR | P value | 95% Cl |
|---|---|---|---|
| LRA | 15.00 | 0.04 | 1.12-202.0 |
| Laboratory | | | |
| Platelets, 1,000/mm3 | 0.95 | 0.13 | 0.96-1.01 |
| Total bilirubin, mg/dL | 1.57 | 0.53 | 0.37-6.53 |

LRA, lung restricted antibody.

### Example 7: The Release of IL1β Increases the Extravasation of LRA

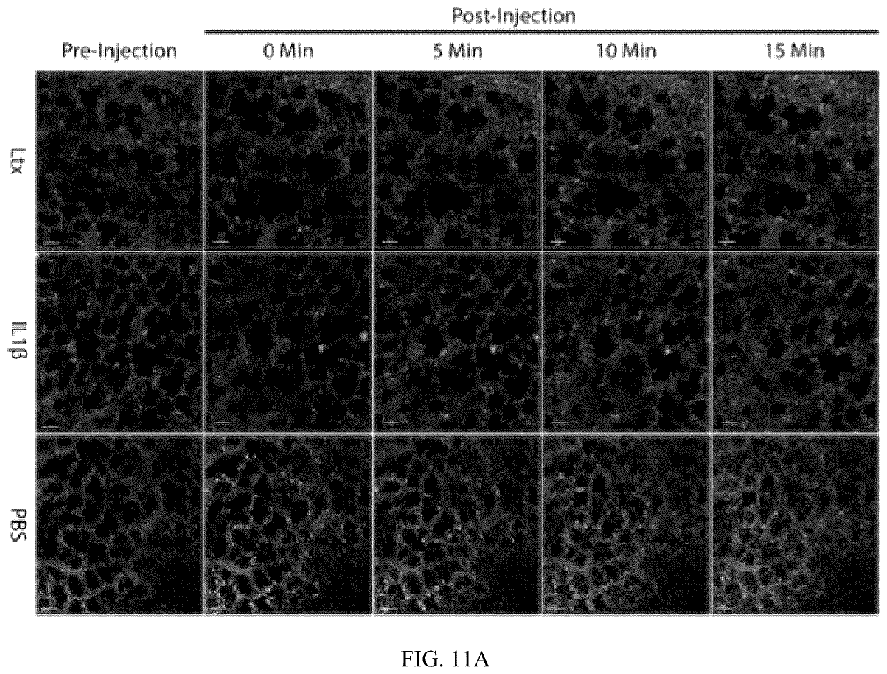

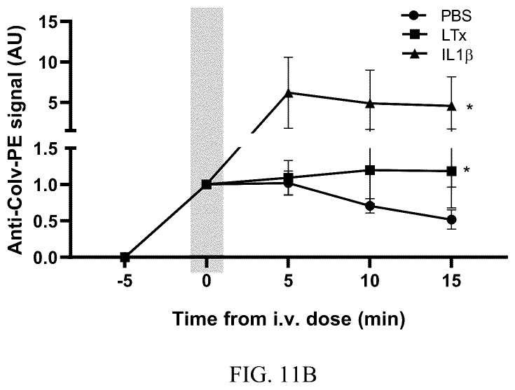

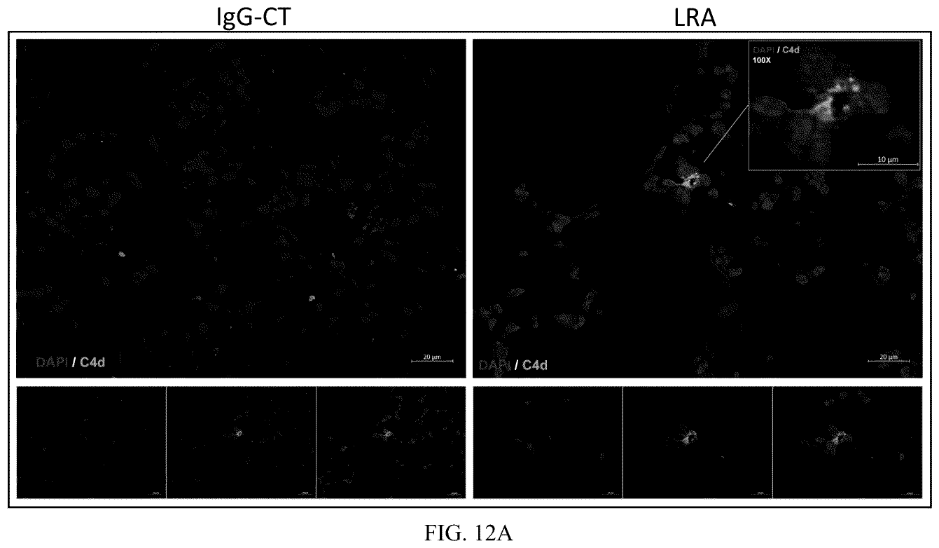

It was previously demonstrated that IL1β facilitates the opening of endothelial gap junctions. This, in turn, enables the extravasation of LRA circulating in the blood. In a subsequent series of experiments, LRA was administered to mice, which then underwent lung transplantation. At the various time points (0 min, 5 min, 10 min, and 15 min), immunohistochemistry and immunofluorescence were conducted to measure the levels of LRA deposited in the lung tissue. In FIG. 11A, LRA (colored pink) was observed to be deposited outside the blood vessels, consistent with prior findings that lung transplantation is associated with the release of IL1β. To further validate the role of IL1β, LRA was administered to normal mice. These mice, now containing LRA, were then given either IL1β or PBS (as a control). Injection with IL1β led to the extravasation of LRA, while PBS showed no effect. FIG. 11A presents the quantification of the extravasation of antibodies following lung transplant, and with treatments of IL1β and PBS.

### Example 8: LRA Activates the Complement System Only after Extravasation

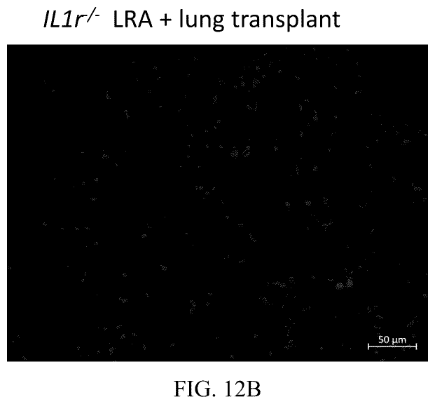

It was observed that LRA induced complement activation specifically after its movement into the extravascular space. In the left panel of FIG. 12A, mice were injected with isotype control antibodies (IgG-CT) and subsequently underwent lung transplantation. The lung allografts were then subjected to complement staining using C4D. No signs of complement activation were detected. However, when mice were administered LRA before undergoing lung transplantation, complement deposition became evident (FIG. 12A, right panel). Notably, when IL1r−/− mice which lack receptors for IL1β received LRA and subsequently underwent lung transplantation, neither complement activation nor LRA extravasation was observed (FIG. 12B)

### Example 9: IL1β is Essential for the Extravasation of LRA

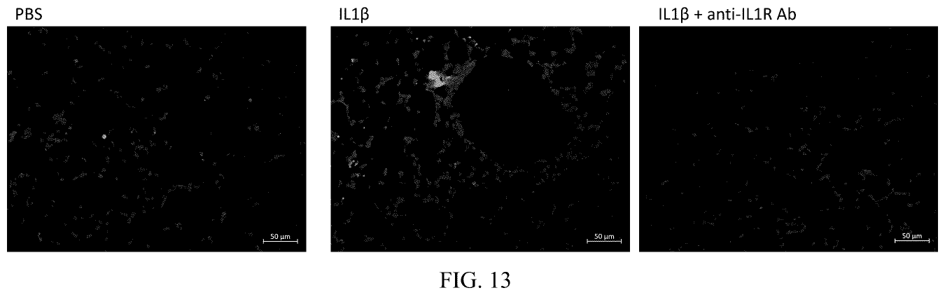

In this study, normal mice were given LRA and subsequently treated with either PBS (serving as a control, FIG. 13, left panel)), IL1β (FIG. 13, middle panel), or a combination of IL1β and anti-IL1R antibodies (FIG. 13, right panel). Through immunofluorescence imaging, it was observed that LRA extravasated and settled when IL1β was administered to the mice (FIG. 13, middle panel). In contrast, in mice treated with both IL1β and antibodies targeting its receptors (IL1R), no extravasation was seen (FIG. 13, right panel). Collectively, these findings underscore the role of IL1β in driving LRA extravasation and the ensuing complement activation. Notably, no complement activation was detected in normal mice. This suggests that although IL1β might leak outside the blood vessels in standard conditions, a lung transplant injury is crucial for antigens to become exposed in the new lungs, allowing LRA binding and complement activation.

### Example 10: Results

Neutrophil-mediated ischemia-reperfusion injury is suggested to be the predominant cause of PGD (1, 25). However, it is evident that the syndrome of PGD includes etiologies other than ischemia-reperfusion injury. Pre-existing LRA are present in a significant proportion of patients undergoing lung transplantation and are strongly associated with an increased risk of PGD (6). Studies in which right and left lungs from single donors were transplanted into different recipients with or without autoantibodies have further suggested their role in the pathogenesis of PGD (26). However, the mechanistic pathways through which LRA contribute to PGD were previously unclear. Importantly, the relationship between LRA-induced graft dysfunction and classical pathways that contribute to transplant-inherent ischemia-reperfusion injury was unknown. The present study identifies clinically actionable pathways through which pre-existing LRA add to ischemia-reperfusion injury to worsen PGD. Given that the LRA caused severe lung graft injury even when neutrophil-mediated ischemia-reperfusion injury was mitigated, it is expected that treatment of LRA-induced lung graft injury could result in amelioration of PGD and improvement in post-transplant outcomes.

The complement system is an essential part of the innate immune system that has been associated with ischemia-reperfusion injury and transplantation (27). Indeed, PGD has been associated with complement deposition although whether these transplant recipients had pre-existing LRA is unknown (28). Additionally, while pre-existing LRA have also been associated with complement deposition in lung grafts, whether they can activate complement remains unclear (26).

The classical complement pathway depends on the binding of C1q protein to antibody attached to antigen, activating C1r and C1s, which cleave C4 and C2. The lectin complement pathway is activated when mannose-binding lectin (MBL) interacts with carbohydrate motifs on pathogens. This results in the activation of MBL-associated proteases which also cleave C4 and C2. Hence, both classical and lectin pathways form C3 convertase after cleaving C4 and C2 and initiate the downstream proteins. In contrast, the alternative pathway is activated by spontaneous hydrolysis of C3, in the presence of Factors B and D, leading to the eventual formation of the C3 and C5 convertase. In the alternative pathway, properdin plays an important role as it stabilizes the protein. All three pathways culminate in the formation of effector compounds (29). Since deletion of mannose-binding protein did not alter the graft injury in LRA-pretreated mice, the lectin pathway is not involved. However, C1q and Factor B inhibition prevented LRA-induced lung graft dysfunction suggesting a role for classical and alternative complement pathways in this model. The findings also explain the immunological mechanisms behind the reported detection of complement proteins in patients with PGD (30). However, the differences with the present study might be related to a focus in LRA, where IgG glycosylation might have a less relevant role than C1q binding to the Fc portion of the IgG. It is also noteworthy that while LRA might activate host cellular immunity and phagocytosis by binding Fcγ receptors on target cells, those did not contribute to the LRA-mediated graft dysfunction since Fcrγ−/−donor lungs experienced similar injury as wild-type grafts. The findings support clinical utility since complement inhibitors are FDA approved and commercially available.

Unlike histocompatibility antigens, the cognate self-antigens for the LRA are non-polymorphic and conserved (31). Ischemia-reperfusion can reveal epitopes of self-antigens which typically serve as structural lung proteins (32), possibly through the activation of matrix metalloproteinases (5, 33-35). Exposure of the self-antigens can then promote the binding with LRA (7). It was previously shown that donor-origin non-classical monocytes retained in the pulmonary vasculature, initiate the pathogenesis of ischemia-reperfusion injury through the recruitment of recipient neutrophils (22, 23). Simultaneously, donor non-classical monocytes activate donor alveolar macrophages which secrete monocyte chemoattractant protein-1, necessary for the mobilization of classical monocytes from the recipient spleen (18, 24). Upon migration to the transplanted lung allograft, the recipient classical monocytes increase the pulmonary vasculature permeability through the release of IL1β, allowing the neutrophils to extravasate and initiate the pathogenesis of ischemia-reperfusion injury (24). Increased endothelial permeability mediated by IL1β was also necessary for the extravasation of LRA into the extravascular space and initiation of LRA induced lung graft injury. Nevertheless, LRA caused lung injury even when donor non-classical monocytes were depleted which results in incomplete recruitment of recipient classical monocytes (18, 24). This may suggest alternative sources of IL1β production or the ability of even low levels of IL1β to cause sufficient endothelial permeability to allow LRA extravasation. Interestingly, treatment with IL1β before LRA injection induced LRA deposition (it facilitates extravasation), but it was not able to induce C4d deposition. These findings will suggest the need for both IL1β ischemia-reperfusion injury to activate the complement and induce C4d deposition (36, 37). Together, these data suggest that while the mechanisms of LRA-induced injury are distinct from ischemia-reperfusion injury, the molecular and immunological changes during ischemia-reperfusion are necessary for the pathogenicity of LRA. The findings also suggest inhibition of IL1β or the IL1β receptor as a therapy to prevent both ischemia-reperfusion and LRA-associated injury. Notably, canakinumab and anakinra, agents that block IL1β or the IL1β receptor, respectively, have relatively benign short-term safety profiles and have received FDA approval for other indications and therefore are suitable for use in treating transplant rejection according to the present disclosure.

Histological features such as alveolar edema, neutrophil infiltration, and capillaritis can be observed in both ischemia-reperfusion injury as well as antibody mediated rejection although presence of complement deposition may favor the former (6, 38-41). Thus, while it is difficult to distinguish between antibody mediated rejection and ischemia-reperfusion injury clinically based on histologic features alone, future prospective studies, potentially including transcriptional profiling (FIG. 2), may distinguish pathological and molecular features to better characterize these disease states and enable delivery of therapies in the right clinical context. Recently, exosomes containing lung-restricted antigens have been detected during PGD in patients containing LRA and could potentially be tested as a biomarker (42). Histology and complement staining may be used, when clinically feasible, to guide the use of complement inhibitors (43). Nevertheless, this should be prospectively validated in clinical trials. Further, future studies incorporating contemporary techniques in proteomics and mass spectroscopy can potentially identify these proteins as well as their immunogenic epitopes.

While only two patients developed features reminiscent of antibody-mediated rejection, their response to treatment combined with our pre-clinical data provides the foundation for a prospective clinical trial. Moreover, while neutrophils are recruited during LRA-induced lung injury above the expected levels during ischemia-reperfusion injury, the mechanisms remain unknown. Donor non-classical monocytes play a dominant role in recruiting host neutrophils through the secretion of chemokines, and it is possible that the LRA-antigen complex may enhance donor non-classical monocytes by binding CD16.

In conclusion, in lung transplant, IL1β increased pulmonary endothelial permeability was found to allow the extravasation of LRA. Binding of LRA to cognate self-antigens in the pulmonary insterstitium and ischemia-reperfusion caused the activation of classical and alternative complement pathways which contributed to the immunopathogenesis of PGD, independent of neutrophil-mediated ischemia-reperfusion injury. C5 inhibiting antibodies as well as Factor B inhibition mitigated LRA-associated lung graft dysfunction in humans and mice.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc.

As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

### REFERENCES

1. Christie J D, Van Raemdonck D, de Perrot M, Barr M, Keshavjee S, Arcasoy S, et al. Report of the ISHLT Working Group on Primary Lung Graft Dysfunction part I: introduction and methods. J Heart Lung Transplant. 2005; 24(10):1451-3.
2. Bharat A, Kuo E, Steward N, Aloush A, Hachem R, Trulock E P, et al. Immunological link between primary graft dysfunction and chronic lung allograft rejection. Ann Thorac Surg. 2008; 86(1):189-97.
3. Morrison M I, Pither T L, and Fisher A J. Pathophysiology and classification of primary graft dysfunction after lung transplantation. J Thorac Dis. 2017; 9(10):4084-97.
4. Wilkey B J, and Abrams B A. Mitigation of Primary Graft Dysfunction in Lung Transplantation: Current Understanding and Hopes for the Future. Semin Cardiothorac Vasc Anesth. 2020; 24(1):54-66.
5. Iwata T, Philipovskiy A, Fisher A J, Presson R G, Jr., Chiyo M, Lee J, et al. Anti-type V collagen humoral immunity in lung transplant primary graft dysfunction. J Immunol. 2008; 181(8):5738-47.
6. Bharat A, Saini D, Steward N, Hachem R, Trulock E P, Patterson G A, et al. Antibodies to self-antigens predispose to primary lung allograft dysfunction and chronic rejection. Ann Thorac Surg. 2010; 90(4):1094-101.
7. Wilkes D S. Autoantibody formation in human and rat studies of chronic rejection and primary graft dysfunction. Semin Immunol. 2012; 24(2):131-5.
8. Bharat A, Chiu S, Zheng Z, Sun H, Yeldandi A, DeCamp M M, et al. Lung-Restricted Antibodies Mediate Primary Graft Dysfunction and Prevent Allotolerance after Murine Lung Transplantation. Am J Respir Cell Mol Biol. 2016; 55(4):532-41.
9. Zaffiri L, Shah R J, Stearman R S, Rothhaar K, Emtiazjoo A M, Yoshimoto M, et al. Collagen type-V is a danger signal associated with primary graft dysfunction in lung transplantation. Transpl Immunol. 2019; 56:101224.
10. Chiu S, Fernandez R, Subramanian V, Sun H, DeCamp M M, Kreisel D, et al. Lung Injury Combined with Loss of Regulatory T Cells Leads to De Novo Lung-Restricted Autoimmunity. J Immunol. 2016; 197(1):51-7.
11. Legoux F P, Lim J B, Cauley A W, Dikiy S, Ertelt J, Mariani T J, et al. CD4+ T Cell Tolerance to Tissue-Restricted Self Antigens Is Mediated by Antigen-Specific Regulatory T Cells Rather Than Deletion. Immunity. 2015; 43(5):896-908.
12. Iwata T, Chiyo M, Yoshida S, Smith G N, Jr., Mickler E A, Presson R, Jr., et al. Lung transplant ischemia reperfusion injury: metalloprotease inhibition down-regulates exposure of type V collagen, growth-related oncogene-induced neutrophil chemotaxis, and tumor necrosis factor-alpha expression. Transplantation. 2008; 85(3):417-26.
13. Haque M A, Mizobuchi T, Yasufuku K, Fujisawa T, Brutkiewicz R R, Zheng Y, et al. Evidence for immune responses to a self-antigen in lung transplantation: role of type V collagen-specific T cells in the pathogenesis of lung allograft rejection. J Immunol. 2002; 169(3):1542-9.
14. Akbarpour M, and Bharat A. Lung Injury and Loss of Regulatory T Cells Primes for Lung-Restricted Autoimmunity. Crit Rev Immunol. 2017; 37(1):23-37.
15. Yanaka S, Yogo R, and Kato K. Biophysical characterization of dynamic structures of immunoglobulin G. Biophys Rev. 2020; 12(3):637-45.
16. Yogo R, Yamaguchi Y, Watanabe H, Yagi H, Satoh T, Nakanishi M, et al. The Fab portion of immunoglobulin G contributes to its binding to Fcgamma receptor III. Sci Rep. 2019; 9(1):11957.
17. Karsten C M, and Kohl J. The immunoglobulin, IgG Fc receptor and complement triangle in autoimmune diseases. Immunobiology. 2012; 217(11):1067-79.
18. Kurihara C, Lecuona E, Wu Q, Yang W, Nunez-Santana F L, Akbarpour M, et al. Crosstalk between nonclassical monocytes and alveolar macrophages mediates transplant ischemia-reperfusion injury through classical monocyte recruitment. JCI Insight. 2021; 6(6):e147282.
19. Serna M, Giles J L, Morgan B P, and Bubeck D. Structural basis of complement membrane attack complex formation. Nat Commun. 2016; 7:10587.
20. Bayly-Jones C, Bubeck D, and Dunstone M A. The mystery behind membrane insertion: a review of the complement membrane attack complex. Philos Trans R Soc Lond B Biol Sci. 2017; 372(1726):20160221.
21. Reichhardt M P, Johnson S, Tang T, Morgan T, Tebeka N, Popitsch N, et al. An inhibitor of complement C5 provides structural insights into activation. Proc Natl Acad Sci USA. 2020; 117(1):362-70.
22. Li W, Terada Y, Tyurina Y Y, Tyurin V A, Bery A I, Gauthier J M, et al. Necroptosis triggers spatially restricted neutrophil-mediated vascular damage during lung ischemia reperfusion injury. Proc Natl Acad Sci USA. 2022; 119(10):e2111537119.
23. Zheng Z, Chiu S, Akbarpour M, Sun H, Reyfman P A, Anekalla K R, et al. Donor pulmonary intravascular nonclassical monocytes recruit recipient neutrophils and mediate primary lung allograft dysfunction. Sci Transl Med. 2017; 9(394):eaal4508.
24. Hsiao H M, Fernandez R, Tanaka S, Li W, Spahn J H, Chiu S, et al. Spleen-derived classical monocytes mediate lung ischemia-reperfusion injury through IL-1beta. J Clin Invest. 2018; 128(7):2833-47.
25. Porteous M K, Diamond J M, and Christie J D. Primary graft dysfunction: lessons learned about the first 72 h after lung transplantation. Curr Opin Organ Transplant. 2015; 20(5):506-14.
26. Fernandez R, Chiu S, Raparia K, Garcha P, Farver C, Budev M, et al. Humoral Human Lung Allograft Rejection by Tissue-Restricted Non-HLA Antibodies. Ann Thorac Surg. 2016; 102(4):e339-41.
27. Howard M C, Nauser C L, Farrar C A, and Sacks S H. Complement in ischaemia-reperfusion injury and transplantation. Semin Immunopathol. 2021; 43(6):789-97.
28. Ngo C, Danel C, Duong-Quy S, Dauriat G, Castier Y, Lortat-Jacob B, et al. C4d detection and histological patterns in the diagnosis of antibody-mediated rejection after lung transplantation: a single-centre study. Histopathology. 2019; 74(7):988-96.
29. Pouw R B, and Ricklin D. Tipping the balance: intricate roles of the complement system in disease and therapy. Semin Immunopathol. 2021; 43(6):757-71.
30. Kulkarni H S, Ramphal K, Ma L, Brown M, Oyster M, Speckhart K N, et al. Local complement activation is associated with primary graft dysfunction after lung transplantation. JCI Insight. 2020; 5(17):e138358.
31. Guerder S, Viret C, Luche H, Ardouin L, and Malissen B. Differential processing of self-antigens by subsets of thymic stromal cells. Curr Opin Immunol. 2012; 24(1):99-104.
32. Yoshida S, Haque A, Mizobuchi T, Iwata T, Chiyo M, Webb T J, et al. Anti-type V collagen lymphocytes that express IL-17 and IL-23 induce rejection pathology in fresh and well-healed lung transplants. Am J Transplant. 2006; 6(4):724-35.
33. Linsenmayer T F, Gibney E, Igoe F, Gordon M K, Fitch J M, Fessler L I, et al. Type V collagen: molecular structure and fibrillar organization of the chicken alpha 1(V) NH2-terminal domain, a putative regulator of corneal fibrillogenesis. J Cell Biol. 1993; 121(5):1181-9.
34. Zheng L, Ward C, Snell G I, Orsida B E, Li X, Wilson J W, et al. Scar collagen deposition in the airways of allografts of lung transplant recipients. American journal of respiratory and critical care medicine. 1997; 155(6):2072-7.
35. Burlingham W J, Love R B, Jankowska-Gan E, Haynes L D, Xu Q, Bobadilla J L, et al. IL-17-dependent cellular immunity to collagen type V predisposes to obliterative bronchiolitis in human lung transplants. J Clin Invest. 2007; 117(11):3498-506.
36. Danobeitia J S, Djamali A, and Fernandez L A. The role of complement in the pathogenesis of renal ischemia-reperfusion injury and fibrosis. Fibrogenesis Tissue Repair. 2014; 7:16.
37. Bongoni A K, Vikstrom I B, McRae J L, Salvaris E J, Fisicaro N, Pearse M J, et al. A potent truncated form of human soluble CR1 is protective in a mouse model of renal ischemia-reperfusion injury. Sci Rep. 2021; 11(1):21873.
38. Levine D J, Glanville A R, Aboyoun C, Belperio J, Benden C, Berry G J, et al. Antibody-mediated rejection of the lung: A consensus report of the International Society for Heart and Lung Transplantation. J Heart Lung Transplant. 2016; 35(4):397-406.
39. Loupy A, and Lefaucheur C. Antibody-Mediated Rejection of Solid-Organ Allografts. N Engl J Med. 2018; 379(12):1150-60.
40. Roux A, Levine D J, Zeevi A, Hachem R, Halloran K, Halloran P F, et al. Banff Lung Report: Current knowledge and future research perspectives for diagnosis and treatment of pulmonary antibody-mediated rejection (AMR). Am J Transplant. 2019; 19(1):21-31.
41. Calabrese F, Hirschi S, Neil D, Montero-Fernandez A, Timens W, Verbeken E, et al. Alveolar septal widening as an “alert” signal to look into lung antibody-mediated rejection: A multicenter pilot study. Transplantation. 2019; 103(11):2440-7.
42. Gunasekaran M, Xu Z, Nayak D K, Sharma M, Hachem R, Walia R, et al. Donor-Derived Exosomes With Lung Self-Antigens in Human Lung Allograft Rejection. Am J Transplant. 2017; 17(2):474-84.
43. Akbarpour M, Wu Q, Liu X, Sun H, Lecuona E, Tomic R, et al. Clinical relevance of lung-restricted antibodies in lung transplantation. Hum Immunol. 2019; 80(8):595-601.
44. Akbarpour M, Lecuona E, Chiu S F, Wu Q, Querrey M, Fernandez R, et al. Residual endotoxin induces primary graft dysfunction through ischemia/reperfusion-primed alveolar macrophages. J Clin Invest. 2020; 130(8):4456-69.
45. Subramanian V, Ramachandran S, Banan B, Bharat A, Wang X, Benshoff N, et al. Immune response to tissue-restricted self-antigens induces airway inflammation and fibrosis following murine lung transplantation. Am J Transplant. 2014; 14(10):2359-66.
46. Young M D, and Behjati S. SoupX removes ambient RNA contamination from droplet-based single-cell RNA sequencing data. Gigascience. 2020; 9(12):giaa51.
47. Wolock S L, Lopez R, and Klein A M. Scrublet: Computational Identification of Cell Doublets in Single-Cell Transcriptomic Data. Cell Syst. 2019; 8(4):281-91.
48. Satija R, Farrell J A, Gennert D, Schier A F, and Regev A. Spatial reconstruction of single-cell gene expression data. Nat Biotechnol. 2015; 33(5):495-502.
49. Aran D, Looney A P, Liu L, Wu E, Fong V, Hsu A, et al. Reference-based analysis of lung single-cell sequencing reveals a transitional profibrotic macrophage. Nat Immunol. 2019; 20(2):163-72.

What is claimed is:

1. A method of preventing or reducing the incidence of primary graft dysfunction in a subject, comprising:
screening the subject for the presence of lung restricted antibodies; and
administering an anti-IL1β antibody and/or complement inhibitor to the subject.

2. The method of claim 1, wherein the administering comprises administering an anti-IL1β antibody.

3. The method of claim 1, wherein the screening step comprises an autoantibody multiplex assay to screen the subject for the lung restricted antibodies.

4. A method of treating or preventing primary graft dysfunction in a subject, comprising: administering an anti-lung restricted antibody.

5. The method of claim 4, wherein the anti-lung restricted antibody comprises an anti-IL1β antibody.

6. The method of claim 6, wherein the anti-IL1β antibody comprises Canakinumab or Anakinra or derivatives thereof.

7. A method of treating or preventing primary graft dysfunction in a subject, comprising: administering a complement inhibitor.

8. The method of claim 7, wherein the complement inhibitor comprises Eculizumab, Cinryze, or Rocunest, or derivatives thereof.

9. The method of claim 7, further comprising administering plasmapheresis and/or a complement blockade.

10. A method of determining the likelihood of primary graft dysfunction in a subject, comprising:
detecting the presence of chemokine IL1β in a sample from the subject following lung transplantation.

11. The method of claim 10, wherein the sample is a blood sample, a tissue sample, or mucous sample.

12. The method of claim 10, wherein the sample is taken from the interstitial space of the lungs.

13. The method of claim 1, wherein the subject is a human being.

14. The method of claim 10, wherein the subject is a human being.
